(19) United States
(12) Patent Application Publication
(10) Pub. No.: US 2017/0129915 A1
(43) Pub. Date: May 11, 2017

(54) THERAPEUTIC AGENT FOR DISEASES ASSOCIATED WITH NERVE AXON DYSFUNCTION, INCLUDING THERAPEUTIC AGENT FOR ALZHEIMER'S DISEASE

(71) Applicant: Resilio Company Limited, Tokyo (JP)

(72) Inventors: Chihiro Tohda, Toyama (JP); Yuji Matsuya, Toyama (JP); Kenji Sugimoto, Toyama (JP)

(21) Appl. No.: 15/306,576

(22) PCT Filed: Apr. 21, 2015
(86) PCT No.: PCT/JP2015/062094

§ 371 Date: Jan. 12, 2017

(30) Foreign Application Priority Data
Apr. 25, 2014 (JP) 2014-091718
Jul. 18, 2014 (JP) 2014-147890

(51) Int. Cl. C07J 43/00; A61K 31/58; A61K 47/44; A23L 33/10

(52) U.S. Cl. CPC C07J 43/006; A23L 33/10; A61K 31/58; A61K 47/44; A23V 2002/00

(57) ABSTRACT

Provided are a clinically applicable drug for radical cure of Alzheimer's disease (AD), and a drug for treating neurological diseases associated with axonal dysfunction other than AD, wherein the drug utilizes the mechanism of action of the AD radical cure therapy. The drugs are oral drugs, each comprising one or more compounds selected from diosgenin, a diosgenin derivative and a pharmaceutically acceptable salt thereof, the one or more compounds being suspended in an edible oil.

### TECHNICAL FIELD

The present invention relates to a clinically applicable drug for preventing or treating diseases associated with the dysfunction of neuronal axons (hereinafter also simply called “axons”). In particular, the present invention relates to a clinically applicable drug for preventing or treating Alzheimer's disease.

### BACKGROUND ART

Alzheimer's disease (hereinafter also called AD) is defined as progressive decline in cognitive function and as dysfunction that is not observed in normal aging process. Diagnostic information of AD is described in the Diagnostic and Statistical Manual of Mental Disorders, 4th Edition (DSM-IV) published by American Psychiatric Association.

Currently, the treatment of AD is limited to symptomatic therapy with use of symptom-improving drugs represented by acetylcholinesterase inhibitors, and drugs for radical cure that are capable of treating the disease itself and stopping the progression thereof have not yet been developed. For the creation of a drug for radical cure of AD, the clarification of the pathogenic mechanism and the development of a new method for controlling the etiologic factors are needed. Cholinergic hypothesis, Aβ hypothesis, tau hypothesis, etc. have been suggested regarding the pathogenic mechanism of AD, and numerous studies are conducted to identify the mechanism.

With the emerging role of acetylcholine in learning and memory, “cholinergic hypothesis of Alzheimer's disease” proposing that degeneration of cholinergic neurons in the basal forebrain and the associated loss of cholinergic neurotransmission in the brain cortex and other areas are largely involved in the deterioration in cognitive function seen in patients with Alzheimer's disease has been argued (Non Patent Literature 1). Based on this action mechanism, acetylcholinesterase inhibitors, which inhibit degradation of acetylcholine in synapses in the brain, are marketed as a therapeutic drug for AD. Examples of the acetylcholinesterase inhibitors include donepezil, galanthamine, and rivastigmine.

Also, Aβ protein, which is a metabolite of amyloid precursor protein (hereinafter also called APP), is considered to be greatly concerned with the degeneration and loss of neurons and the expression of cognitive deficits (Non Patent Literature 2 and 3). β secretase and γ secretase participate in the formation of Aβ proteins, and depending on the cleavage site of the protein, Aβ (1-38), which consists of 38 amino acids, Aβ (1-40) having two more amino acids at the C terminus, Aβ (1-42) having four more amino acids at the C terminus, etc. are produced. These Aβs are highly aggregative (Non Patent Literature 4) and are primary constituents of the senile plaques (Non Patent Literature 4, 5, 6, and 7). That is, such aggregates eventually change into insoluble precipitates and highly-concentrated neuritic plaques, which are the pathological features of AD (Non Patent Literature 8). Further, mutations in APP and presenilin genes observed in familial AD are known to increase these Aβ proteins (Non Patent Literature 9, 10, and 11). Therefore, compounds capable of lowering the Aβ production are expected to be promising drugs that retard progression of AD or prevent AD. Based on the expectation, creation of drugs, such as Aβ antibodies and secretase inhibitors, intended to lower the Aβ production has been attempted. Some candidate therapeutic drugs for AD based on the hypothesis are currently under clinical trials, and a certain level of efficacy in AD patients has been reported (Non Patent Literature 12 and 13).

As described above, the drugs currently used for AD patients in clinical practice can prevent or retard the onset or progression of AD, but cannot improve the cognitive function. That is, the current treatment of AD is limited to symptomatic therapy with use of symptom-improving drugs represented by acetylcholinesterase inhibitors, and there has not been developed drugs for radical cure that are capable of improving the disease itself. For the creation of a drug for radical cure of AD, the development of a method for controlling the causative factors of neurological dysfunction is needed. In addition, providing a compound that is suited to the new mechanism is truly desired.

Non Patent Literature 15 and 16 reported intraperitoneal administration of diosgenin enhanced the memory of normal or AD model mice. The literature also reported that the enhancement of memory was due to the extension of axons. From the report, diosgenin is expected to be effectively used for radical cure of AD. All the examinations on the effects of diosgenin in the literature were performed through intraperitoneal administration. However, in the case of the application to, for example, humans, intraperitoneal administration is not clinically practical.

### CITATION LIST

### Non Patent Literature

Non patent literature 1: Francis, P et al., J Neurol Neurosurg Psychiatry 66, 137-147, 1999.
Non patent literature 2: Klein, W L et al., Proceeding of the National Academy of Science USA, September, 2, 100(18), 10417-10422, 2003.
Non patent literature 3: Nitsch, R M et al., Neuron, May 22, 38, 547-554, 2003.
Non patent literature 4: Jarrett, J T et al., Biochemistry, 32(18), 4693-4697, 1993.
Non patent literature 5: Glenner, G G et al., Biochemical and Biophysical Research Communications, May 16, 120(3), 885-890, 1984.
Non patent literature 6: Masters, C L et al., Proceeding of the National Academy of Science USA, June, 82(12), 4245-4249, 1985.
Non patent literature 7: Gong, Y et al., PNAS 100, 10417-10422, 2003.
Non patent literature 8: Hardy, J et al., Science 297, 353-356, 2002.
Non patent literature 9: Gouras, G K et al., American Journal of Pathology, January, 156(1), 15-20, 2000.
Non patent literature 10: Scheuner, D et al., Nature Medicine, 1996, August, 2(8), 864-870.
Non patent literature 11: Forman, M S et al., Journal of Biological Chemistry, December, 19, 272(51), 32247-32253, 1997.
Non patent literature 12: Mount, C et al., Nature Medicine 12, 780-784, 2006.
Non patent literature 13: Siemers, E R et al., Clinical Neuropharmacology, 30, 317-325, 2007.
Non patent literature 14: Cho, S et al., Experimental Neurology, 203, 274-278, 2007.
Non patent literature 15: SCIENTIFIC REPORTS, Volume 2, Number 535, 1-11.
Non patent literature 16: Tohda C, Lee Y A, Goto Y, Nemere I. Sci Rep. 2013 Dec. 2, 3:3395. 1-8.

### SUMMARY OF INVENTION

### Technical Problem

In view of the above problems, a principal object of the present invention is to create a clinically applicable drug for radical cure of AD. Another principal object of the present invention is to provide a drug for treating neurological diseases associated with axonal dysfunction other than AD, wherein the drug utilizes the mechanism of action of the AD radical cure therapy. Other objects will become apparent from the description of the present specification.

### Solution to Problem

The inventors conducted extensive studies to solve the above problems and, as a result, found that oral administration of a diosgenin solution in an aqueous solvent (a mixture of an organic solvent and water) fails to confer the memory enhancing effect of diosgenin, whereas, unexpectedly, oral administration of a diosgenin suspension in an oil or fat effectively confers the memory enhancing effect of diosgenin (the inventors also found that oral administration of the suspension effectively confers the memory enhancing effect at a low dose as compared with the intraperitoneal administration) Based on these useful, significant new findings specific to the present invention, the inventors performed further studies and found that, in addition to oral administration of such a diosgenin suspension, oral administration of a suspension of a diosgenin derivative compound in an oil or fat also achieves significant memory enhancing effect. Based on this useful, new finding specific to the present invention, the inventors further conducted examinations and completed the present invention.

That is, the present invention relates to the following.

[1] An oral drug comprising one or more compounds selected from diosgenin, a diosgenin derivative [a compound derived from diosgenin by substitution at the C3 hydroxyl group (such as an amino acid-substituted derivative, an aminosulfonic acid-substituted derivative, a carbamate-substituted derivative, and a halogenated derivative)] and a pharmaceutically acceptable salt thereof, the one or more compounds being suspended or dissolved in an oil or fat.
[2] The drug according to the above [1], comprising at least diosgenin.
[3] The drug according to the above [1] or [2], wherein the diosgenin derivative is at least one compound selected from a compound represented by formula (I-1):

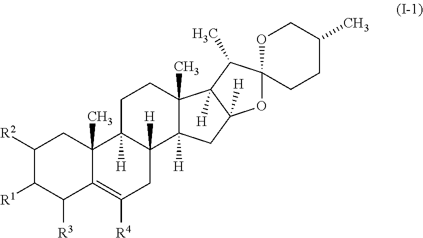

(wherein R¹, R², R³, and R⁴ are the same or different and each are a hydrogen atom or a substituent, with the proviso that when R², R³, and R⁴ are a hydrogen atom, R¹ is not a hydroxyl group) and
a pharmaceutically acceptable salt thereof.
[4] The drug according to the above [3], wherein the substituent in formula (I-1) is a hydrocarbon group, a hydroxyl group, an —O—(CH₂)ₙ—CH₃ group, an —O—(CH₂)ₘ—NH₂ group, an —O—(CH₂)ₘ—COOH group, an —O—(CH₂)ₘ—SO₃H group, an —O—CO—(CH₂)ₙ—CH₃ group, an —O—CO—NH—(CH₂)ₙ—CH₃ group, an —O—CO—NR—(CH₂)ₙ—CH₃ group, an —O—CO—NH—CH(R^{b})—COOH group, an —O—(CH₂)ₙ—CO—NH-AD group (wherein AD is an adamantyl group), an —O—CO—NH—(CH₂)ₘ—SO₃H group, an —O—CO—NH—(CH₂)ₘ—COOH group, an —O—CO—O—(CH₂)ₘ—CH₃ group, an —O—CO—S—(CH₂)ₙ—CH₃ group, an —O—SU group (wherein SU is a sugar chain), an —O—SO₂—OH group, an —O—PO₂—OH group, an —(OCH₂CH₂)ₘ—CH₃ group, an —(OCH₂CH₂CH₂)ₘ—CH₃ group, a carboxyl group, a —COO(CH₂)ₙCH₃ group, a —CO—NH—(CH₂)ₙ—CH₃ group, an —SO₃H group, an —SO₂—(CH₂)ₙ—CH₃ group, an —SO₂-Ph group (wherein Ph is a phenyl group), a —CO—NH—CH(R^{b})—COOH group, a —CO—NH—(CH₂)ₙ—SO₃H group, an amino group, an —NH—(CH₂)ₙ—CH₃ group, an —NH—(CH₂)ₙ—NH₂ group, an —NH—CH(R^{b})—COOH group, an —NH—(CH₂)ₘ—SO₃H group, an —NH—(CH₂)ₘ—SO₂H group, an —NH—CO—O—(CH₂)ₙ—CH₃ group, an —NH—CO—NH₂ group, an —NH—CO—NH-AD group (wherein AD is an adamantyl group), an —NH—CO—NH—CH(R^{b})—COOH group, an —NH—CO—NH—(CH₂)ₘ—SO₃H group, an —NH—CO—NH—(CH₂)ₘ—COOH group, a mercapto group, an —S—(CH₂)ₙ—CH₃ group, an —S—(CH₂)ₘ—COOH group, an —S—(CH₂)ₘ—CH(NH₂)—COOH group, an —S—CO—NH-AD group (wherein AD is an adamantyl group), an —S—S—(CH₂)ₘ—CH(NH₂)—COOH group, an —SO₃H group, a —PO₃H group, an amino acid group, or a halogen atom (in the above formulae m is an integer of 1 or more, n is an integer of 0 or more, and R^{b} is a hydrogen atom or a hydrocarbon group).
[5] The drug according to any one of the above [1] to [4], wherein the diosgenin derivative is one or more compounds selected from the group consisting of (3β,25R)-3-(2-aminoethanoyloxy)-spirost-5-ene, (3β,25R)-3-fluorospirost-5-ene, (3β,25R)-3-(2-aminoethylsulfonyloxy)-spirost-5-ene, (3β,25R)-3-(2-aminopropylsulfonyloxy)-spirost-5-ene, (3β,25R)-3-[N-(2,6-dimethyladamantan-1-yl)carbamoyloxy]-spirost-5-ene, (3β,25R)-3-{[N-(2,6-dimethyladamantan-1-yl)carbamoyl]amino}-spirost-5-ene, (3β,25R)-3-[N-(2, 6-dimethyladamantan-1-yl)carbamoylthio]-spirost-5-ene, (3β,25R)-3-{[N-(adamantan-1-yl)carbamoyl]amino}-spirost-5-ene, (3β,25R)-3-[N-(adamantan-1-yl)carbamoylthio]-spirost-5-ene, (3β,25R)-3-[N-(adamantan-1-yl)carbamoyloxy]-spirost-5-ene, and pharmaceutically acceptable salts thereof.
[6] The drug according to any one of the above [1] to [5], which is a prophylactic or therapeutic drug for a disease associated with axonal dysfunction.
[7] The drug according to the above [6], wherein the disease associated with axonal dysfunction is Alzheimer's disease.
[8] The drug according to the above [6], wherein the disease associated with axonal dysfunction is spinal cord injury.
[9] The drug according to the above [1] to [5], which is a drug for extending axons.
[10] The drug according to the above [1] to [5], which is a drug for repairing degenerated axons.
[11] The drug according to the above [1] to [5], which is a drug for improving (enhancing) memory or a drug for suppressing (or preventing) the deterioration of memory (for example, age-related deterioration of memory).
[12] The drug according to any one of the above [1] to [11], which is combined with one or more compounds known to be effective for the treatment or prevention of a disease associated with axonal dysfunction or a pharmaceutically acceptable salt thereof.
[13] The drug according to the above [1] to [12], which is in one or more dosage forms selected from the group consisting of liquids, suspensions, capsules, soft capsules, tablets, granules, powders, syrups, jellies, orally disintegrating tablets, and chewable tablets.
[14] A functional health food comprising the drug according to any one of the above [1] to [13].
[15] (3β,25R)-3-fluorospirost-ene represented by formula (III):

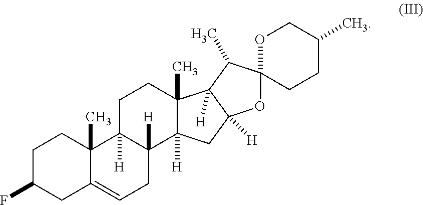

The present invention also includes (1) a method for preventing and/or treating a disease associated with axonal dysfunction, (2) a method for extending axons, (3) a method for repairing degenerated axons, or (4) a method for improving (or enhancing) memory or a method for suppressing (or preventing) the deterioration of memory (for example, age-related deterioration of memory), each method comprising administering the above oral drug to an animal including a human.

The present invention also includes a novel diosgenin derivative (for example, a compound represented by formula (III)).

The present invention also includes a prophylactic or therapeutic drug for a disease associated with axonal dysfunction, the drug comprising at least one compound selected from a diosgenin derivative (for example, a compound represented by formula (I-1)) and a pharmaceutically acceptable salt thereof. The disease may be Alzheimer's disease or spinal cord injury, in particular, spinal cord injury.

The present invention further includes a drug for extending axons, a drug for repairing degenerated axons, a drug for improving (enhancing) memory, or a drug for suppressing (or preventing) the deterioration of memory (for example, age-related deterioration of memory), each drug comprising at least one compound selected from a diosgenin derivative and a pharmaceutically acceptable salt thereof.

The present invention further includes a medicament (or a pharmaceutical composition) comprising at least one compound selected from a diosgenin derivative and a pharmaceutically acceptable salt thereof.

The present invention further includes a functional health food comprising at least one compound selected from a diosgenin derivative and a pharmaceutically acceptable salt thereof.

The present invention further includes (1) a method for preventing and/or treating a disease associated with axonal dysfunction, (2) a method for extending axons, (3) a method for repairing degenerated axons, or (4) a method for improving (or enhancing) memory or a method for suppressing (or preventing) the deterioration of memory (for example, age-related deterioration of memory), each method comprising administering, to an animal including a human, at least one compound selected from a diosgenin derivative and a pharmaceutically acceptable salt thereof (i.e., the above drug comprising at least one compound selected from a diosgenin derivative and a pharmaceutically acceptable salt thereof, the above medicament comprising at least one compound selected from a diosgenin derivative and a pharmaceutically acceptable salt thereof, or the above functional health food comprising at least one compound selected from a diosgenin derivative and a pharmaceutically acceptable salt thereof).

The inventors also found that a diosgenin derivative has a stimulating activity on 1,25D₃-MARRS and that such a substance with a stimulating activity on 1,25D₃-MARRS is effectively used for the prevention and/or treatment of neurological diseases (including the above-exemplified diseases associated with axonal dysfunction, such as Alzheimer's disease and spinal cord injury).

Hence the present invention also includes the following.

[A] A diosgenin derivative for preventing and/or treating a neurological disease.
[B] The diosgenin derivative according to the above [A], wherein the neurological disease is Alzheimer's disease, dementia, Parkinson's disease, spinal cord injury, or brain contusion.
[C] The diosgenin derivative according to the above [A] or [B] in the production of a prophylactic and/or therapeutic drug for a neurological disease.
[D] The use according to the above [C], wherein the neurological disease is Alzheimer's disease, dementia, Parkinson's disease, spinal cord injury, or brain contusion.
[E] The diosgenin derivative according to the above [A] for use in the prevention and/or treatment of a neurological disease.
[F] The diosgenin derivative for use according to the above [E], wherein the neurological disease is Alzheimer's disease, dementia, Parkinson's disease, spinal cord injury, or brain contusion.
[G] A pharmaceutical composition for treating a neurological disease, the composition comprising a therapeutically effective amount of the diosgenin derivative according to the above [A].
[H] The pharmaceutical composition according to the above [F], wherein the neurological disease is Alzheimer's disease, dementia, Parkinson's disease, spinal cord injury, or brain contusion.
[I] The pharmaceutical composition according to the above [G] or [H], further comprising a therapeutically effective amount of one or more compounds known to be effective for the treatment or prevention of a disease or a pharmaceutically acceptable salt thereof.
[J] The pharmaceutical composition according to the above [I], wherein the one or more compounds known to be effective for the treatment or prevention of a disease or a pharmaceutically acceptable salt thereof is one or more compounds known to be effective for the treatment or prevention of a neurological disease or a pharmaceutically acceptable salt thereof.
[K] A method for preparing the pharmaceutical composition according to any one of the above [H] to [J], the method comprising adding at least one carrier.
[L] A method for preventing and/or treating a neurological disease, the method comprising administering the diosgenin derivative according to the above [A] to an animal including a human.
[M] The method according to the above [L], wherein the administration of the diosgenin derivative according to the above [A] is combined with administration of one or more compounds known to be effective for the treatment or prevention of a neurological disease or a pharmaceutically acceptable salt thereof.
[N] The method according to the above [L] or [M], wherein the neurological disease is Alzheimer's disease, dementia, Parkinson's disease, spinal cord injury, or brain contusion.
[O] A kit for preventing and/or treating a neurological disease, the kit comprising the diosgenin derivative according to the above [A].
[P] The kit according to the above [O], comprising the diosgenin derivative according to the above [A] and a container.
[Q] A method for activating 1,25D₃-MARRS, the method comprising administering a diosgenin derivative.
[R] A method for preventing or treating Alzheimer's disease, the method comprising administering a diosgenin derivative.
[S] A method for reducing amyloid plaques, tau deposition, tau precipitates, PHF-tau, or neurofibrillary tangles, the method comprising administering a diosgenin derivative to a mammal including a human.
[T] A method for suppressing Aβ (1-42)-induced axonal atrophy, the method comprising administering a diosgenin derivative to a mammal including a human.
[U] A method for activating a signaling pathway through stimulation of 1,25D₃-MARRS, the method comprising administering a diosgenin derivative to a mammal including a human.
[V] A health food, a functional food, or a specified health food, the food comprising a diosgenin derivative.

### Advantageous Effects of Invention

The present invention provides a clinically applicable drug for radical cure of AD. The present invention also provides a prophylactic or therapeutic drug for diseases associated with axonal dysfunction other than AD. The present invention further provides a drug for extending axons and a drug for repairing degenerated axons.

### BRIEF DESCRIPTION OF DRAWINGS

FIG. 1 shows the results of an object recognition memory test (Examples 1 to 4 and Comparative Example 1).

FIG. 2 shows the results of an object recognition memory test (Examples 5 and 6 and Comparative Example 2).

FIG. 3 shows the results of an object recognition memory test (Examples 7 and 8 and Comparative Example 3).

FIGS. 4A and 4B show the results of a hindlimb motor function evaluation test using spinal cord injury model mice (Example 10 and Comparative Example 5). FIG. 4A shows the Basso Mouse Scale (BMS) scores, and FIG. 4B shows the Toyama Mouse Scale (TMS) scores.

FIG. 5A shows the results of a spontaneous motor activity test in Reference Test 1, and FIG. 5B shows the results of weight measurement in Reference Test 1.

FIGS. 6A and 6B show the results of Reference Test 2. FIG. 6A shows the results of Reference Example 1, and FIG. 6B shows the results of Reference Example 2.

FIG. 7 shows the results of Reference Test 3.

FIG. 8 shows the results of an object recognition memory test (Example 11 and Comparative Example 6).

FIG. 9 shows the results of an object recognition memory test (Comparative Examples 7 and 8).

FIG. 10 shows the results of an object recognition memory test (Example 12 and Comparative Example 9).

### DESCRIPTION OF EMBODIMENTS

The present invention will be described in detail below.

An embodiment of the present invention relates to an oral drug comprising one or more compounds selected from diosgenin, a diosgenin derivative, and a pharmaceutically acceptable salt thereof.

The term “one or more compounds selected from diosgenin, a diosgenin derivative, and a pharmaceutically acceptable salt thereof” herein may be abbreviated as “diosgenin or the like”.

Diosgenin is a steroid sapogenin represented by formula (I):

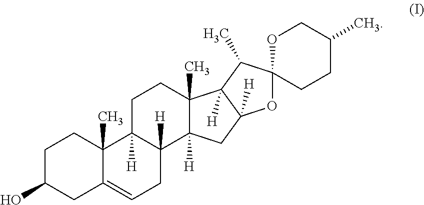

Diosgenin is known to present in some kinds of plants, such as herbal plants, including Dioscorea rhizome, Trigonella spp., Polygonatum spp., Smilax spp., etc. Diosgenin has been reported to have various effects, such as anticancer effect (Yan, L. L. et al., Exp Oncol, 31, 27-32, 2009), anti-food allergy effect (Huang, C. H. et al., Planta Med, 75, 1300-1305, 2009), suppressing effect on oxidative stress-induced memory deficits caused by galactose administration (Chiu, C. S. et al., Am J Chin Med, 39, 551-563, 2011) and anti-diabetic neuropathy (Kang, T. H. et al., Biol Pharm Bull, 34, 1493-1498, 2011). In addition, diosgenin is known for skin whitening effect (JP 2010-535758 W), skin improvement effect including wrinkle removal (JP 2009-501209 W and JP 2007-016013 A), hair growth effect (JP 2006-273754 A), etc.

The diosgenin usable in the present invention is not particularly limited as long as the effects of the invention are not impaired. The diosgenin may be a commercially available product, a product produced by a known or conventional method or an equivalent method, or an extract from a natural product.

The diosgenin derivative herein is a compound that may be a diosgenin equivalent. The diosgenin derivative to be used may be a commercially available product, a product produced by a known or conventional method or an equivalent method, or an extract from a natural product. For example, the diosgenin derivative may be a diosgenin equivalent obtainable by a chemical modification of diosgenin, for example, substituent introduction or substituent conversion, or may be a diosgenin glycoside (dioscin etc.) extracted from a natural product.

The diosgenin derivative is not particularly limited, and specific examples thereof include a compound derived from diosgenin by substitution at the C3 hydroxyl group, a compound derived from diosgenin by substitution at the C2 position (or a compound derived from diosgenin by replacement of the C2 hydrogen atom by a substituent), a compound derived from diosgenin by substitution at the C4 position (or a compound derived from diosgenin by replacement of the C4 hydrogen atom by a substituent), a compound derived from diosgenin by substitution at the C6 position (or a compound derived from diosgenin by replacement of the C6 hydrogen atom by a substituent), and a salt thereof. Other specific examples of the diosgenin derivative include a derivative derived by esterification of the C3 hydroxyl group (for example, an amino acid-substituted derivative, an aminosulfonic acid-substituted derivative, and a carbamate-substituted derivative) and a derivative derived by halogenation of the C3 hydroxyl group.

The diosgenin derivative (and a salt thereof) is exemplified by a compound represented by formula (I-1) and a (pharmaceutically acceptable) salt thereof.

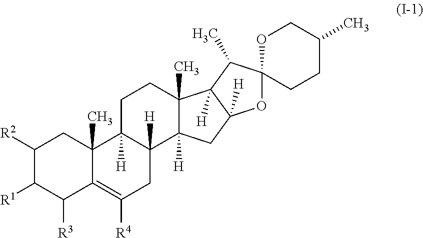

(In the formula, R¹, R², R³, and R⁴ are the same or different and each are a hydrogen atom or a substituent, with the proviso that when R², R³, and R⁴ each are a hydrogen atom, R¹ is not a hydroxyl group.)

The substituent at the position of R¹ is exemplified by hydrocarbon groups {for example, saturated or unsaturated aliphatic hydrocarbon groups including alkyl groups [including straight or branched alkyl groups (for example, C₁₋₁₂ alkyl groups, preferably C₁₋₈ alkyl groups) such as a methyl group, an ethyl group, an n-propyl group, an isopropyl group, an n-butyl group, an isobutyl group, an s-butyl group, a t-butyl group, and a pentyl group], cycloalkyl groups (for example, C₄₋₁₀ cycloalkyl groups, preferably C₅₋₈ cycloalkyl groups, such as a cyclopentyl group, a cyclohexyl group, a cycloheptyl group, and a cyclooctyl group), aralkyl groups (for example, C₆₋₁₀ aryl C₁₋₄ alkyl groups such as a benzyl group and a phenethyl group), and polycyclic aliphatic hydrocarbon groups (such as a decalinyl group, a norbornyl group, an adamantyl group, and a dimethyladamantyl group); and aromatic hydrocarbon groups including aryl groups (for example, C₆₋₁₀ aryl groups such as a phenyl group, a tolyl group, and a xylyl group)}; hetero atom-containing groups (including nitrogen atom-containing groups, oxygen atom-containing groups, sulfur atom-containing groups, and phosphorus atom-containing groups) {for example, oxygen atom-containing groups [such as a hydroxyl group, an oxo group (═O), an —OR^{a} group, an —O—CO—R^{a} group, an —O—CO—N(R^{b})₂ group, an —O—CO—O—R^{a} group, an —O—CO—S—R^{a} group, an —OR^{c} group, an —O—SO₂—OH group, an —O—PO₂—OH group, an —(OR^{d})ₖ—R^{e} group, a carboxyl group, a —CO—O—R^{a} group, and a —CO—N(R^{b})₂ group], nitrogen atom-containing groups [such as an amino group, an —NR^{a}R^{b} group, an —NR^{b}—CO—O—R^{a} group, an —NR^{b}—CO—N(R^{b})₂ group, and nitrogen-containing cyclic groups (including groups corresponding to pyridine, pyrroline, pyrrole, indole, and the like)], sulfur atom-containing groups [such as a mercapto group, an —SR^{a} group, an —S—S—R^{a} group, a sulfo (—SO₃H) group, an —SO₂—R^{b} group, and an —S—CO—N(R^{b})₂ group], phosphorus atom-containing groups [such as a phosphate (H₂PO₄—) group and a —PO₃H group], and amino acid groups [or amino acid residues, for example, a group formed by esterification between the hydroxyl group at the 3 position of diosgenin (corresponding to the substitution position of R¹ in the above formula) and a carboxyl group of an amino acid (such as glycine and alanine)]}; and halogen atoms (such as a fluorine atom, a chlorine atom, a bromine atom, and an iodine atom).

In the above formulae, R^{a} is a hydrocarbon group (including the above-exemplified hydrocarbon groups such as an alkyl group); R^{b} is a hydrogen atom or a hydrocarbon group (including the above-exemplified hydrocarbon groups such as an alkyl group); R^{c} is a sugar (or a sugar chain or a sugar residue); R^{d} is an alkylene group (including C₂₋₄ alkylene groups such as an ethylene group, a propylene group, and a trimethylene group); R^{e} is a hydrogen atom, a hydroxyl group, or a hydrocarbon group (including the above-exemplified hydrocarbon groups such as an alkyl group (for example, a methyl group)); and k is an integer of 2 or more (for example, 2 to 10). R^{a} and R^{b} may be the same or different groups, and when a plurality of R^{b}s are present, R^{b}s may be the same or different.

When R^{a} and R^{b} are each a hydrocarbon group (such as an alkyl group), R^{a} and R^{b} may have a substituent. Examples of the substituent include, but are not limited to, the above-exemplified substituents, including oxygen atom-containing groups (such as a hydroxyl group, a carboxyl group, an —OR^{a} group, and an —O—CO—R^{a} group), nitrogen atom-containing groups (such as an amino group and an —NR^{a}R^{b} group), and sulfur atom-containing groups (such as a mercapto group, an —SR^{a} group, a sulfo group, and an —SO₂—R^{b} group).

The hydrocarbon group may have a single substituent as exemplified above or two or more of such substituents in combination.

When the hydrocarbon group has substituents, the number of substituents may be 1 or more and is, for example, 1 to 10 (for example, 1 to 8), preferably 1 to 6 (for example, 1 to 4), and more preferably about 1 to 3.

When R¹ is a substituent, R¹ is typically exemplified by hydrocarbon groups [including alkyl groups (such as a —(CH₂)ₙ—CH₃ group), cycloalkyl groups, and aralkyl groups], hetero atom-containing groups {including oxygen atom-containing groups [such as a hydroxyl group, an —O—(CH₂)ₙ—CH₃ group, an —O—(CH₂)ₘ—NH₂ group, an —O—(CH₂)ₘ—COOH group, an —O—(CH₂)ₘ—SO₃H group, an —O—CO—(CH₂)ₙ—CH₃ group, an —O—CO—NH—(CH₂)ₙ—CH₃ group, an —O—CO—NR—(CH₂)ₙ—CH₃ group, an —O—CO—NH—CH(R^{b})—COOH group, an —O—(CH₂)ₙ—CO—NH-AD group (where AD is an adamantyl group (such as a 1-adamantyl group and a 2,6-dimethyladamantan-1-yl group)), an —O—CO—NH—(CH₂)ₘ—SO₃H group, an —O—CO—NH—(CH₂)ₘ—COOH group, an —O—CO—O—(CH₂)ₙ—CH₃ group, an —O—CO—S—(CH₂)ₙ—CH₃ group, an —O—SU group (where SU is a sugar chain), an —O—SO₂—OH group, an —O—PO₂—OH group, a —(OCH₂CH₂)ₘ—CH₃ group, an —(OCH₂CH₂CH₂)ₘ—CH₃ group, a carboxyl group, a —COO(CH₂)ₙCH₃ group, a —CO—NH—(CH₂)ₙ—CH₃ group, an —SO₃H group, an —SO₂—(CH₂)ₙ—CH₃ group, an —SO₂-Ph group (where Ph is a phenyl group), a —CO—NH—CH(R^{b})—COOH group, and a —CO—NH—(CH₂)ₙ—SO₃H group], nitrogen atom-containing groups [such as an amino group, an —NH—(CH₂)ₙ—CH₃ group, an —NH—(CH₂)ₙ—NH₂ group, an —NH—CH(R^{b})—COOH group, an —NH—(CH₂)ₘ—SO₃H group, an —NH—(CH₂)ₘ—SO₂H group, an —NH—CO—O—(CH₂)ₙ—CH₃ group, an —NH—CO—NH₂ group, an —NH—CO—NH-AD group (where AD is an adamantyl group (such as a 1-adamantyl group and a 2,6-dimethyladamantan-1-yl group)), an —NH—CO—NH—CH(R^{b})—COOH group, an —NH—CO—NH—(CH₂)ₘ—SO₃H group, and an —NH—CO—NH—(CH₂)ₘ—COOH group], sulfur atom-containing groups [such as a mercapto group, an —S—(CH₂)ₙ—CH₃ group, an —S—(CH₂)ₘ—COOH group, an —S—(CH₂)ₘ—CH(NH₂)—COOH group, an —S—CO—NH-AD group (where AD is an adamantyl group (such as a 1-adamantyl group and 2,6-dimethyladamantan-1-yl group)), an —S—S—(CH₂)ₘ—CH(NH₂)—COOH group, and an —SO₃H group], phosphorus atom-containing groups [such as a —PO₃H group], and amino acid groups (such as an —O—CO—CH₂—NH₂ group)}, and halogen atoms (such as a fluorine atom, a chlorine atom, a bromine atom, and an iodine atom).

In the above formulae, m is an integer of 1 or more (for example, 1 to 10, preferably 1 to 4, more preferably 1 or 2); n is an integer of 0 or more (for example, 0 to 10, preferably 0 to 7); and R^{b} is as defined above [i.e., a hydrogen atom or a hydrocarbon group (such as an alkyl group)].

The substituents at the positions of R², R³, and R⁴ are the same as those exemplified for R¹.

When R² and/or R⁴ is a substituent, the substituent is typically exemplified by oxygen atom-containing groups, nitrogen atom-containing groups, sulfur atom-containing groups, amino acid groups, and halogen atoms.

When R³ is a substituent, the substituent is typically exemplified by halogen atoms.

In formula (I-1), the combination of the substituents at the positions of R¹ to R⁴ is not limited, and all possible combinations are included. The combination of the substituents at the positions of R¹ to R⁴ is typically exemplified by the following.

(1) A combination of substituents in which R¹ is a substituent other than a hydroxyl group and R² to R⁴ each are a hydrogen atom.
(2) A combination of substituents in which R¹ is a substituent other than a hydroxyl group, R² is a substituent, and R³ and R⁴ each are a hydrogen atom.
(3) A combination of substituents in which R¹ is a substituent other than a hydroxyl group, R³ is a substituent, and R² and R⁴ each are a hydrogen atom.
(4) A combination of substituents in which R¹ is a substituent other than a hydroxyl group, R² and R³ each are a substituent, and R⁴ is a hydrogen atom.
(5) A combination of substituents in which R¹ is a substituent other than a hydroxyl group, and R², R³ and R⁴ each are a substituent.
(6) A combination of substituents in which R¹ is a substituent other than a hydroxyl group, R² and R³ each are a hydrogen atom, and R⁴ is a substituent.
(7) A combination of substituents in which R¹ is a substituent other than a hydroxyl group, R³ is a hydrogen atom, and R² and R⁴ each are a substituent.
(8) A combination of substituents in which R¹ is a substituent other than a hydroxyl group, R² is a hydrogen atom, and R³ and R⁴ each are a substituent.
(9) A combination of substituents in which R¹ is a hydroxyl group, R² is a substituent, and R³ and R⁴ each are a hydrogen atom.
(10) A combination of substituents in which R¹ is a hydroxyl group, R³ is a substituent, and R² and R⁴ each are a hydrogen atom.
(11) A combination of substituents in which R¹ is a hydroxyl group, R² and R³ each are a substituent, and R⁴ is a hydrogen atom.
(12) A combination of substituents in which R¹ is a hydroxyl group, and R² to R⁴ each are a substituent.
(13) A combination of substituents in which R¹ is a hydroxyl group, R² and R³ each are a hydrogen atom, and R⁴ is a substituent.
(14) A combination of substituents in which R¹ is a hydroxyl group, R² is a hydrogen atom, and R³ and R⁴ each are a substituent.
(15) A combination of substituents in which R¹ is a hydroxyl group, R³ is a hydrogen atom, and R² and R⁴ each are a substituent.

Specific examples of the diosgenin derivative include, but are not limited to, (3β,25R)-3-(2-aminoethanoyloxy)-spirost-5-ene represented by formula (II):

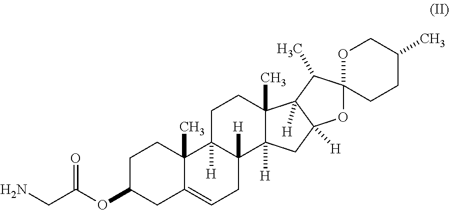

and
a pharmaceutically acceptable salt thereof, (3β,25R)-3-fluorospirost-5-ene represented by Chemical formula (III):

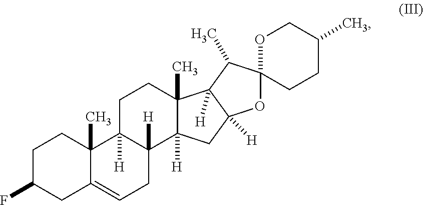

(3β,25R)-3-(2-aminoethylsulfonyloxy)-spirost-5-ene, (3β,25R)-3-(2-aminopropylsulfonyloxy)-spirost-5-ene, (3β,25R)-3-[N-(2, 6-dimethyladamantan-1-yl) carbamoyloxy]-spirost-5-ene, (3β,25R)-3-{[N-(2,6-dimethyladamantan-1-yl)carbamoyl]amino}-spirost-5-ene, (3β,25R)-3-[N-(2,6-dimethyladamantan-1-yl)carbamoylthio]-spirost-5-ene, (3β,25R)-3-{[N-(adamantan-1-yl) carbamoyl]amino}-spirost-5-ene, (3β,25R)-3-[N-(adamantan-1-yl)carbamoylthio]-spirost-5-ene, and (3β,25R)-3-[N-(adamantan-1-yl)carbamoyloxy]-spirost-5-ene [or (3β,25R)-3-(1-adamantyl-aminocarbonyloxy)-spirost-5-ene].

The diosgenin derivative (or a salt thereof) may be a commercially available product or a product synthesized by a known method. For example, in cases where a substituent is introduced into the position of R¹, the substituent can be introduced via the hydroxyl group originally present on diosgenin (the hydroxyl group at the 3 position). In cases where a halogen atom is introduced into the position of R² or R³, an approach that may be adopted involving converting (oxidizing) R¹ into an oxo group and then halogenating a carbon adjacent to the resulting ketone to introduce a halogen to the position of R² or R³ (as necessary, the oxo group is further converted (reduced) into a hydroxyl group). In cases where a halogen atom is introduced to the position of R⁴, the halogen introduction can be achieved via, for example, electrophilic halogenation on an unsaturated bond. In addition, various substituents can be introduced by using a nucleophilic reagent containing a hetero atom (such as an oxygen atom, a nitrogen atom, and a sulfur atom).

The “pharmaceutically acceptable salt (or salt)” herein is not particularly limited and includes a pharmaceutically acceptable salt of diosgenin or the like. Specific examples of the salt include hydrogen halides (for example, hydrofluoride, hydrochloride, hydrobromide, hydroiodide, etc.), inorganic acid salts (for example, sulfate, nitrate, perchlorate, phosphate, carbonate, bicarbonate, etc.), organic carboxylates (for example, acetate, oxalate, maleate, tartrate, fumarate, citrate, etc.), organic sulfonates (for example, methanesulfonate, trifluoromethanesulfonate, ethanesulfonate, benzenesulfonate, toluenesulfonate, camphorsulfonate, etc.), amino acid salts (for example, aspartate, glutamate, etc.), organic amine salts (for example, salts with organic bases, such as trimethylamine, triethylamine, pyridine, picoline, dicyclohexylamine, N,N′-dibenzylethylenediamine, arginine and lysine), quaternary amine salts, alkali metal salts (for example, sodium salt, potassium salt, etc.), alkaline earth metal salts (for example, magnesium salt, calcium salt, etc.), and the like.

The one or more compounds selected from diosgenin, a diosgenin derivative, and a pharmaceutically acceptable salt thereof (hereinafter may be abbreviated as diosgenin or the like) contained in the oral drug of the present invention is preferably provided in the form of a suspension in an oil or fat. In the course of developing the present invention, the inventors unexpectedly found that oral administration of a suspension of diosgenin or the like in an oil or fat confers markedly high pharmaceutical efficacy of diosgenin or the like.

The “oil or fat” in the present invention is not necessarily in a liquid form when orally administered, and may be in the form of a liquid, a semi-solid, or a solid. The oil or fat herein includes edible oils, oils and fats used as a vehicle, an excipient, an emulsifier, or the like in pharmaceutical products, and oily pharmaceutical products.

Alternatively, a solution of diosgenin or the like dissolved in an oil or fat may be used in the present invention.

The edible oil usable in the present invention is not particularly limited as long as the effects of the invention are not impaired. Examples of the edible oil include vegetable oils, such as soybean oil, rapeseed oil (canola oil), high oleic rapeseed oil, corn oil, sesame oil, sesame salad oil, unroasted sesame oil, perilla oil, linseed oil, peanut oil, safflower oil, high oleic safflower oil, sunflower oil, high oleic sunflower oil, cottonseed oil, grape seed oil, macadamia nut oil, hazelnut oil, peanut oil, almond oil, nut oil, walnut oil, pumpkin seed oil, walnut oil, lemon oil, camellia oil, tea seed oil, perilla seed oil, borage oil, olive oil, rice oil, rice bran oil, wheat germ oil, palm oil, palm olein, palm stearin, palm kernel oil, coconut oil, and cacao butter; animal oils and fats, such as beef tallow, lard, chicken fat, milk fat, and fish oil (for example, sardine oil, mackerel oil, cod oil, whale oil, and cod liver oil); fatty acids (such as docosahexaenoic acid (DHA) and eicosapentaenoic acid (EPA)); and lipid-soluble vitamins (such as vitamin A and vitamin E). Examples of the oil or fat used in pharmaceutical products include, in addition to the above edible oils, medium-chain triglycerides and esters of iodinated poppy-seed oil fatty acid. These oils and fats may be used singly or in combination of two or more of them.

Suspending of diosgenin or the like in an oil or fat can be done by any method in the present invention, and may be performed by a known or conventional method for suspending a compound (a water-soluble compound or a lipid-soluble compound) in an oil or fat or an equivalent method thereof. Specifically, suspending of diosgenin or the like may be done by, for example, adding an oil or fat to diosgenin or the like and then stirring the mixture with a homogenizer or the like.

In an embodiment of the present invention, the ratio of the amount of diosgenin or the like to the amount of an oil or fat in a suspension of diosgenin or the like in an oil or fat is not particularly limited as long as the effects of the invention are achieved. The amount of diosgenin or the like in terms of mole per unit volume (mL) of the oil or fat may be, for example, typically about 1 nmol/mL to about 1,000 nmol/mL. For improvement of the bioavailability of diosgenin or the like, the amount is preferably about 10 nmol/mL to about 100 nmol/mL. Also in the case of a solution of diosgenin or the like dissolved in an oil or fat, the ratio of the amount of diosgenin or the like to the amount of an oil or fat may be as defined above.

The dosage form of the oral drug of the present invention is not particularly limited and may be any dosage form that allows diosgenin or the like to be suspended in an oil or fat. Examples of the dosage form include liquids, suspensions, capsules, soft capsules, tablets, granules, powders, syrups, jellies, orally disintegrating tablets, and chewable tablets. These dosage forms can be produced by a conventional method.

The oral drug of the present invention may further comprise, in addition to diosgenin or the like and an oil or fat, a stabilizer, an emulsifier, a suspending agent, a surfactant, a pH adjuster, a buffering agent, an antiseptic, a colorant, a flavor, an odor corrective, and the like, as desired.

Examples of the stabilizer include, but are not limited to, an antioxidant (such as ascorbic acid, tocopherol, sorbic acid, and retinol) and a chelating agent (such as edetic acid, citric acid, tartaric acid, and salts thereof).

Examples of the emulsifier include, but are not limited to, benzalkonium chloride, glycerol, propylene glycol, cetanol, lecithin, lanolin, and sodium lauryl sulfate.

Examples of the suspending agent include, but are not limited to, gum arabic, benzalkonium chloride, kaolin, carmellose, sodium lauryl sulfate, lauryl aminopropionic acid, glycerol monostearate, polyvinyl alcohol, polyvinylpyrrolidone, sodium carboxymethyl cellulose, methyl cellulose, hydroxymethyl cellulose, hydroxyethyl cellulose, and hydroxypropyl cellulose.

Examples of the surfactant include, but are not limited to, polysorbates (such as polysorbate 20, polysorbate 40, polysorbate 60, polysorbate 65, and polysorbate 80), polyoxyethylene-polyoxypropylene copolymers, polyoxyethylene hydrogenated castor oil, sorbitan monostearate, and sodium lauryl sulfate.

Examples of the buffering agent include, but are not limited to, phosphoric acid salts, carbonic acid salts, acetic acid salts, citric acid salts, and lactic acid salts.

Examples of the pH adjuster include, but are not limited to, inorganic acids such as hydrochloric acid and phosphoric acid, organic acids such as acetic acid, citric acid, and lactic acid, inorganic bases such as sodium hydroxide, potassium hydroxide, and sodium carbonate, and organic bases such as meglumine and trometamol.

Examples of the antiseptic include, but are not limited to, p-hydroxybenzoic acid esters, chlorobutanol, benzyl alcohol, phenethyl alcohol, dehydroacetic acid, and sorbic acid.

Examples of the colorant include, but are not limited to, edible pigments, β-carotene, and riboflavin.

Examples of the flavor include, but are not limited to, lemon oil, orange oil, menthol, and peppermint oil.

Examples of the odor corrective include, but are not limited to, citric acid, adipic acid, ascorbic acid, fructose, D-sorbitol, glucose, saccharin sodium, simple syrup, sucrose, honey, sweet hydrangea leaf, licorice, citric acid, adipic acid, ascorbic acid, orange oil, orange peel tincture, fennel oil, peppermint, and menthol.

Diosgenin or the like as the active ingredient of the oral drug of the present invention preferably has an effect of extending axons and/or repairing degenerated axons.

SCIENTIFIC REPORTS, Volume 2, Number 535, pp 1-11 describes that diosgenin stimulates 1,25D₃-MARRS to promote axons extension and that the axon extension effect in turn enhances a memory. The effects of diosgenin or the like used in the present invention may also be based on a similar mechanism of action.

In an embodiment of the present invention, the oral drug is a prophylactic or therapeutic drug for a disease associated with axonal dysfunction. Examples of the disease associated with axonal dysfunction include, but are not limited to, spinal cord injury, brain contusion, Alzheimer's disease (AD), Parkinson's disease, and dementia. The term “dementia” in the present invention includes cerebrovascular dementia, Lewy body dementia, frontotemporal dementia, Pick's disease, and the like, but excludes Alzheimer's disease. The oral drug is particularly preferably a prophylactic or therapeutic drug for AD or spinal cord injury.

The amount of diosgenin or the like as the active ingredient of the oral drug of the present invention is not particularly limited, but is preferably a sufficient amount for the treatment, improvement, alleviation, or resolution of the symptoms associated with the disease.

The dosage of the oral drug of the present invention may be appropriately set depending on, for example, the severity of the symptoms, the age, sex, and body weight of a subject of administration, the mode of administration, the type of salt, the type of disease, and the like, and the dosage of the oral drug is not particularly limited. The dosage of the oral drug in terms of the molar amount of diosgenin or the like as the active ingredient per unit body weight of a subject of administration may be, for example, typically about 0.001 to about 1,000 μmol/kg·day, preferably about 0.01 to about 10 μmol/kg·day, and more preferably 0.01 to about 1 μmol/kg·day.

The oral drug of the present invention exhibits sufficient effects even when administered in a relatively small dosage. For example, the dosage described in Non Patent Literature 15 and 16 is 10 μmol/kg·day, whereas the dosage in the present invention can be smaller than those described in the literature. That is, the dosage in the present invention can be less than 10 μmol/kg·day (for example, 5 μmol/kg·day or less), preferably 3 μmol/kg·day or less (for example, 0.001 to 2 μmol/kg·day), more preferably 1 μmol/kg·day or less (for example, 0.003 to 0.5 μmol/kg·day), and particularly preferably 0.3 μmol/kg·day or less (for example, 0.005 to 0.2 μmol/kg·day).

The daily dosage may be given as a single dose or divided into several doses.

The subject of administration of the oral drug of the present invention is not particularly limited but is preferably mammals including a human. The mammals including a human are not particularly limited and examples thereof include humans, monkeys, hamadryas baboons, chimpanzees, mice, rats, guinea pigs, hamsters, rabbits, cats, dogs, sheep, goats, pigs, cattle, and horses.

In an embodiment of the present invention, the oral drug may be used in combination with one or more compounds known to be effective for the treatment or prevention of a disease associated with axonal dysfunction or a pharmaceutically acceptable salt thereof.

In a preferred embodiment of the present invention, when the disease associated with axonal dysfunction is AD, the oral drug of the present invention may further comprise, in addition to diosgenin or the like, one or more compounds known to be effective for the treatment or prevention of AD or the symptoms thereof. In another preferred embodiment of the present invention, the oral drug of the present invention may be used in combination with a pharmaceutical composition comprising one or more compounds known to be effective for the treatment or prevention of AD or the symptoms thereof.

When the oral drug is used in combination with one or more compounds or composition, the form of the combination is not particularly limited and may be, for example, a drug combination or a product combination.

Examples of the compound known to be effective for the treatment or prevention of AD or the symptoms thereof include compounds having the following action mechanisms for the treatment of diseases caused by amyloid β (Aβ), for example, AD, senile dementia, Down's syndrome, amyloidosis, etc.

Specific examples thereof include compounds having the action mechanism of choline esterase inhibitors (for example, donepezil, huperzine A, tacrine, rivastigmine, and galantamine); AMPA receptor antagonists (for example, 1,2-dihydropyridine compounds, such as 3-(2-cyanophenyl)-5-(2-pyridyl)-1-phenyl-1,2-dihydropyridin-2-one); NMDA receptor antagonists (for example, memantine); acetylcholine release stimulants (for example, pramiracetam and aniracetam); calcium channel agonists (for example, nefiracetam); free radical scavengers (for example, EGb 761); platelet activator antagonists (for example, EGb 761); platelet aggregation antagonists (for example, EGb 761 and triflusal); insulin sensitizers (for example, rosiglitazone); peroxisome proliferator-activated receptor agonists (for example, rosiglitazone); peroxisome proliferator-activated receptor γ agonists (for example, rosiglitazone); monoamine oxidase B inhibitors (for example, rasagiline, selegiline, and procaine); carnitine acetyltransferase stimulants (for example, levacecarnine); NSAIDs (for example, cyclooxygenase-2 inhibitors, such as triflusal and celecoxib); nerve growth factor agonists (for example, xaliproden and FPF 1070); β amyloid inhibitors (for example, tarenflurbil, tramiprosate, and leuprorelin-D); immunomodulators (for example, tarenflurbil, immune globulin, and icosapent ethyl ester); NF-κB inhibitors (for example, tarenflurbil); thyrotropin-releasing hormones (for example, taltirelin); dopamine D2 receptor antagonists (for example, risperidone); serotonin 2 receptor antagonists (for example, risperidone); muscarinic M1 receptor agonists (for example, cevimeline); α1-adrenergic receptor agonists (for example, modafinil); serotonin 3 receptor antagonists (for example, alosetron); dopamine D2 receptor agonists (for example, aripiprazole); dopamine D2 receptor antagonists (for example, aripiprazole); serotonin 1A receptor agonists (for example, aripiprazole); serotonin 2A receptor antagonists (for example, aripiprazole); glucocorticoid antagonists (for example, mifepristone); progesterone antagonists (for example, mifepristone); HMG-CoA reductase inhibitors (for example, atorvastatin and simvastatin); adenosine uptake inhibitors (for example, propentofylline); phosphodiesterase inhibitors (for example, propentofylline); acetylcholine receptor agonists (for example, choline alfoscerate); membrane-permeation enhancers (for example, choline alfoscerate); cannabinoid 1 receptor antagonists (for example, rimonabant); cannabinoid receptor agonists (for example, dronabinol); angiogenesis inhibitors (for example, paclitaxel); immunosuppressants (for example, paclitaxel); tubulin antagonists (for example, paclitaxel); thromboxane A synthase inhibitors (for example, triflusal); antioxidants (for example, idebenone); a adrenergic receptor antagonists (for example, nicergoline); estrogen antagonists (for example, conjugated estrogens and trilostane); 3-β-hydroxysteroid dehydrogenase inhibitors (for example, trilostane); signaling pathway inhibitors (for example, trilostane); melatonin receptor agonists (for example, ramelteon); immunostimulants (for example, immune globulin, icosapent ethyl ester, and procaine); HIV entry inhibitors (for example, procaine); sodium channel antagonists (for example, procaine); microtubule inhibitors (for example, CPH 82); glycine NMDA agonists (for example, cycloserine); adenosine A1 receptor antagonists (for example, KW 3902); ATPase stimulants (for example, triacetyluridine); mitochondrial functional enhancers (for example, triacetyluridine); growth hormone-releasing factor agonists (for example, tesamorelin); butyl choline esterase inhibitors (for example, bisnorcymserine); α adrenergic receptor antagonists (for example, nicergoline); NO synthase type II inhibitors (for example, arundic acid); chelating agents (for example, PBT 2); amyloid fibril formation inhibitors (for example, TTP488, PF 4494700); serotonin 4 receptor agonists (for example, PRX 03140); serotonin 6 receptor antagonists (for example, SB 742457); benzodiazepine receptor inverse agonists (for example, radequinil); Ca channel antagonists (for example, safinamide); nicotine receptor agonists (for example, ispronicline); BACE inhibitors (for example, CTS 21166); or the like.

Further, specific examples of the compounds include Cilostazol, donepezil, huperzine A, tacrine, rivastigmine, galantamine, pramiracetam, aniracetam, nefiracetam, Egb 761, rosiglitazone, rasagiline, levacecarnine, celecoxib, 3-(2-cyanophenyl)-5-(2-pyridyl)-1-phenyl-1,2-dihydropyridin-2-one, talampanel, becampanel, memantine, xaliproden, tarenflurbil, tramiprosate, leuprorelin-D, taltirelin, risperidone, cevimeline, modafinil, alosetron, aripiprazole, mifepristone, atorvastatin, propentofylline, choline alfoscerate, FPF 1070 (CAS No. 143637-01-8), rimonabant, dronabinol, docosahexaenoic acid, paclitaxel, triflusal, idebenone, nicergoline, conjugated estrogens, trilostane, simvastatin, selegiline, ramelteon, immune globulin, icosapent ethyl ester, procaine, CPH 82, cycloserine, KW 3902 (CAS No. 136199-02-5), triacetyluridine, estrogen dementia therapeutics (e.g., MIGENIX, Vancouver, Canada), tesamorelin, bisnorcymserine, nicergoline, arundic acid, PBT 2, TTP488, PF 4494700, PRX 03140, SB 742457, radequinil, safinamide, ispronicline, CTS 21166, bapineuzumab, NP 031112, (2S,3aS,7aS)-1-{[(R,R)-2-phenylcyclopropyl]carbonyl}-2-[(thiazolidin-3-yl)carbonyl]octahydro-1H-indole, citalopram, venlafaxine, leuprorelin, prasterone, peptide T (CAS No. 53-43-0), besipiridine, lexipafant, stacofylline, SGS 742 (CAS No. 123690-78-8), T 588 (CAS No. 142935-03-3), nerispiridine, dexanabinol, sabcomeline, GTS 21 (CAS No. 156223-05-1), CX516 (CAS No. 154235-83-3), ABT 089 (CAS No. 161417-03-4), anapsos, tesofensine, SIB 1553A (i.e., 4-[[2-(1-methyl-yl-2-pyrrolidinyl)ethyl]thia]phenol), ladostigil, radequinil, GPI 1485, ispronicline, arundic acid, MEM 1003 (i.e., 3-isopropyl 5-(2-methoxyl) 4-(2-chloro-3-cyanophenyl)-2,6-dimethylpyridine-3,5-dicarboxylase), V 3381 (i.e., 2-(2,3-dihydro-1H-inden-3-ylamino)acetamide hydrochloride), farampator, paliroden, prasterone-paladin, urocortin, DP b99 (i.e., 2,2′-(ethylenedioxy)bis(2, 1-phenylene)bis[N-[2-[2-(octyloxy)ethoxy]-2-oxoethyl]imino]bis(acetic acid)), capserod, DU 125530, bapineuzumab, AL 108 (i.e., L-Asparaginyl-L-alanyl-L-prolyl-L-valyl-L-seryl-L-isoleucyl-L-prolyl-L-glutamine), DAS 431, DEBIO 9902, DAR 100, mitoquinone, IPL 455903 (i.e., 5(S)-[3-(cyclopenthyloxy)-4-methoxyphenyl]-3(S)-(3-methylbenzyl)piperidin-2-one), E2CDS, PYM 50028, PBT 2, lecozotan, SB 742457, CX 717, AVE 1625 (i.e., 1-(bis(4-chlorophenyl)methyl)-3-((3,5-difluorophenyl)(methylsulfonyl)methylene)azetidine), LY 450139 (i.e., N2-[2(s)-hydroxy-3-methylbutyryl]-N1-[3-methyl-2-oxo-2,3,4, 5-tetrahydro-1H-3-benzazepin-1(S)-yl]-L-alaninamide), EM 1421 (i.e., 4,4′-[(2R,3S)-2,3-dimethylbutane-1,4-diyl]bis(1,2-dimethoxy benzene), SRN 001, TTP488, PRX 03140, dimebolin, glycine-proline-glutamate, C105, AL 208, MEM 3454, AC 1202, L 830982, LY 451395 (i.e., (R)—N-[2-[4′-(methylsulfonamidemethyl)biphenyl-4-yl]propyl]propane-2-sulfonamide), MK 0249, LY 2062430, diethylnorspermine, neboglamine, S 18986, SA 4503 (CAS No. 165377-44-6), GRI 1, S 17092 (i.e., (2S,3aS,7aS)-1-{[(R,R)-2-phenylcyclopropyl]carbonyl}-2-[(thiazolidin-3-yl)carbonyl]octahydro-1H-indole), SL 251188, EUK 189, R 1450, 6,6-dimethyl-3-(2-hydroxyethyl)thio-1-(thiazol-2-yl)-6,7-dihydro-2-benzothiophen-4(5H)-one, CERE 110, dexefaroxan, CAD 106, HF 0220, HF 0420, EHT 0202, VP 025,

MEM 1414, BGC 201259 (i.e., N,N-dimethylcarbamic acid, 4-[1(S)-(methylamino)-3-(4-nitrophenoxy)propyl]phenyl ester), EN 100, ABT 834, ABT 239 (i.e., 4-[2-[2-[(2R)-2-methylpyrrolidinyl]ethyl]-benzofuran-5-yl]benzonitrile), SGS 518, R 1500, C 9138, SSR 180711, alfatradiol, R 1577, T 817MA (i.e., 1-[3-[2-(1-benzothiophen-5-yl)ethoxy]propyl]azetidin-3-ol maleate), CNP 1061 (i.e., 4-methyl-5-(2-nitrooxyethyl)thiazole), KTX 0101 (i.e., β-hydroxybutyric acid sodium salt), GSK 189254 (i.e., 6-[3-cyclobutyl-2,3,4,5-tetrahydro-1H-benzo[d]azepin-7-yloxy]-N-methylnicotinamide), AZD 1080, ACC 001, PRX 07034, midazolam, R-phenserine, AZD 103 (CAS No. 488-59-5), SN 522, NGX 267 (CAS No. 503431-81-0), N-PEP-12, RN 1219, FGLL, AVE 8112, EVT 101, NP 031112, MK 0752, MK 0952, LX 6171, PAZ 417, AV965, PF 3084014, SYN 114, GSI 953, SAM 315, SAM 531, D-serine, leteprinim potassium, BR 16A (CAS No. 149175-77-9), RPR 107393 (CAS No. 190841-57-7), NXD 2858, REN 1654, CDD 0102, NC 1900 (CAS No. 132925-74-7), ciclosporin, NCX 2216 (i.e., (E)-4-(nitrooxy)butyl-3-[4-[2-(2-fluorobiphenyl-4-yl)propanoyloxy]-3-methoxypheny]acrylate), NXD 3109, NXD 1191, ZSET 845 (i.e., 3,3-diphenylimidazo[1,2-a]pyridin-2-(3H)-one), ET 002, NT 13, RO 638695 (i.e., [1,6-(1,6-dioxohexyl)]dipyrrolidine-(2R)-carboxylic acid), bisnorcymserine, BA 1016, XD 4241, EUK 207 (i.e., (SP-5-13)-(acetato-κO) [13,16,19,22-tetraoxa-3,6-diazatricyclo[21.3.18,12]octacosa-1(27),2,6,8,10,12(28),23,25-octaene-27,28-diolate(2-)-κN3, κN6, κO27, κO28] magnesium salt), LG 617 inhibitors, ZSET 1446, PAN 811, F 14413 (i.e., 2-[5-fluoro-2 (S)-methoxy-2,3-dihydro-1,4-benzodioxin-2-yl]-4,5-dihydro-1H-imidazole), FP 7832 (i.e., N-[2-(5-methoxy-1-nitroso-1H-indol-3-yl)ethyl]acetamide), ARA 014418 (i.e., N-(4-methoxybenzyl)-N′-(5-nitro-1,3-thiazol-2-yl)urea), AZD 3102, KP 544 (i.e., 2-amino-5-(4-chlorophenylethynyl)-4-(4-trans-hydroxycyclohexylamino)pyrimidine), DP 155, 5-chloro-N-[3-[2-(dimethylamino)ethyl]-1H-indol-5-yl]naphthalene-2-sulfonamide, TAK 070,
huperzine, N-[2-(3,5-dimethyladamant-1-yl)ethyl]acetamide hydrochloride, 6-[4-[(dimethylamino)methyl]-5-ethyl-2-methoxypheny]pyridin-2-amine, 4,6-diphenyl-3-(4-(pyrimidin-2-yl)piperazin-1-yl)pyridazine, N-[(1S,2R)-3-(3, 5-difluorophenyl)-1-hydroxy-1-[(5S,6R)-5-methyl-6-(neopentyloxy)molpholin-3-yl]propan-2-yl]acetamide hydrochloride, N-[(1R,2S)-3-(3,5-difluorophenyl)-1-hydroxy-1-[(2R,4R)-4-phenoxypyrrolidin-2-yl]propan-2-yl]-3-[(R)-2-(methoxymethyl)pyrrolidine-1-carbonyl]-5-methylbenzamide, R 1589, midafotel, phenserine, coluracetam, physostigmine, cipralisant, nitroflurbiprofen, PPI 1019 (i.e., 3α,5β,7α,12α)-trihydroxycholan-24-oyl-L-leucyl-L-valyl-L-phenylalanyl-L-phenylalanyl-L-alanine), dapsone, MDL 100453 (CAS No. 129938-34-7), NS 377, midaxifylline, propofol phosphate, metrifonate, ceronapril, tenilsetam, sufoxazine, seglitide, ebiratide, nebracetam, milacemide, iododoxorubicin, SM 10888 (CAS No. 129297-21-8), U 80816 (CAS No. 138554-11-7), YM 954 (CAS No. 132041-85-1), SUT 8701 (CAS No. 123577-73-1), apovincamine, FR 121196 (CAS No. 133920-65-7), LY 274614 (CAS No. 136109-04-1), CL 275838 (CAS No. 115931-65-2), igmesine, K 7259 (CAS No. 133667-88-6), vinconate, itasetron, CL 287663 (CAS No. 125109-98-0), WAY 100289 (CAS No. 136013-69-9), SR 46559A (CAS No. 137733-33-6), GYKI 46903 (CAS No. 142999-59-5), L 670548 (CAS No. 121564-89-4), Y 29794 (CAS No. 129184-48-1), AF 125 (CAS No. 7631-86-9), KFM 19 (CAS No. 133058-72-7), ST 796 (i.e., (S)-3-[3-(trifluoromethyl)benzoyl)amino]hexahydroazepin-2-one, RU 33965 (CAS No. 122321-05-5), SDZ 210086 (i.e., (−)-1′,2(S)-dimethylspiro[1,3-dioxane-4,4′-piperidine]), L 689660 (CAS No. 144860-79-7), L 689560 (CAS No. 139051-78-8), ST 618 (i.e., 1-(6,7-dimethoxy-1,2,3,4-tetrahydro-2-naphthyl)-4-hydroxypyrrolidin-2-one), U 74500A (CAS No. 110101-65-0), GEA 857 (CAS No. 120493-42-7), BIBN 99 (CAS No. 145301-48-0), DX 9366, ONO 1603 (CAS No. 114668-76-7), MDL 102234 (CAS No. 137766-81-5), P 9939 (CAS No. 157971-37-4), PD 140532 (CAS No. 157971-39-6), azetirelin, MR 16728 (CAS No. 147614-21-9), dabelotine, MDL 102503 (i.e., 8-[1(R)-methyl-2-phenylethyl]-1,3-dipropyl-7H-xanthine), PD 141606 (i.e., (±)-(Z)-3-(3-phenyl-2-propynyloxyimino)-1-azabicyclo[2.2.1]heptane), SNK 882 (CAS No. 152221-12-0), L 696986 (CAS No. 141553-45-9), tazomeline, LY 235959 (CAS No. 137433-06-8), 2-(2-thioxopyrrolidin-1-yl)acetamide, AK 30 NGF, ABT 418 (CAS No. 147402-53-7), itameline, HUP 13, sibopirdine, KST 5452 (CAS No. 157998-88-4), TJ 54, U 92798 (i.e., 7-[4-[bis(4-fluorophenyl)methyl]perhydro-1,4-diazepin-1-ylmethyl]-4-isopropyl-2-methoxy-2,4,6-cycloheptatrien-1-one), U 92032 (CAS No. 142223-92-5),
3-(sulfamoyloxy)estra-1,3,5(10)-trien-17-one, P 11012 (CAS No. 164723-36-8), A 82695 (CAS No. 147388-86-1), FR 76659 (CAS No. 116904-25-7), apaxifylline, CX 417, 7 MEOTA (CAS No. 5778-80-3), BU 4514N (CAS No. 151013-39-7), pregnenolone, mexidol, ST 857 (CAS No. 154755-63-2), RU 49041 (CAS No. 123828-80-8), RU 35929 (CAS No. 111711-47-8), P 878184, P 128 (CAS No. 157716-52-4), eurystatin A, eurystatin B, LK 12, NBI 108, NBI 107, NBI 117, L 705106, bacoside A+B, clausenamide, SM 21 (CAS No. 155156-22-2), alaptide, RS 17017 (i.e., 1-(4-amino-5-chloro-2-methoxypheny)-5-(1-piperidinyl)-1-pentanone hydrochloride), AF 150 (S) (i.e., (S)-[1-methyl-piperidine-4-spiro-(2′-methylthiazoline)]), RO 153505 (CAS No. 78771-13-8), PV 113 (i.e., 1,2,3,4-tetrahydropyrrole-[1,2-a]-pyrazine), arisugacin, A 98284 (i.e., 2(R)-(3-methylxazol-5-yl)quinuclidine), AP 5 (CAS No. 136941-85-0), BD 1054, SDZ NDD 094 (i.e., bis-(2-(2-methylimidazol-1-yl]methyl)-pyridine-tris(hydrogen-fumarate), AZ 36041 (CAS No. 173324-76-0), quilostigmine, A 84543 (i.e., 3-[1-methylpyrrolidin-2-(S)-ylmethoxy]pyridine fumarate), BTG 4247 (i.e., (2-[2-chloroethoxy[4-(dimethylamino)phenyl]phosphoryl]-acetohydrazine), CGP 50068 (CAS No. 158647-49-5), cerebrocrast, desferri-nordanoxamine, isolichenan, MHP 133 (i.e., 3-(N, N-dimethoxycarbamoyloxy)-1-methyl-2-(4-phenyl-semicarbazomethyl)pyridium chloride), FR 152558 (CAS No. 151098-08-7), GVS 111 (CAS No. 157115-85-0), P 11149 (CAS No. 164724-79-2), PDC 008004, KST 2818 (CAS No. 158623-26-8), KST 5410 (CAS No. 158623-27-9), RU 52583 (CAS No. 123829-33-4), PD 151832 (CAS No. 149929-39-5), UCL 1199 (i.e., 4-[2-[(5-nitropyridin-2-ylsulfanil)ethyl]-1H-imidazole), isovanihuperzine A, SIB 1765F (CAS No. 179120-52-6), JWS USC 751X (i.e., 3-[[[2-[[(5-dimethylaminoethyl)-2-furanyl]methyl]thio]ethy]amino]-4-nitropyridazine), GR 175737 (i.e., 3-(4-chlorobenzyl)-5-[2-(1H-imidazol-4-yl)ethyl]-1,2,4-oxadiazole), KS 505A (CAS No. 131774-53-3), ZTTA 1 (i.e., N-benzyloxycarbonyl-thiopropyl-thiopropynal-dimethylacetal), AGN 190837 (CAS No. 136527-40-7), P 10358 (188240-59-7), WAY 131256 (CAS No. 174001-71-9), DBO 83 (i.e., 3-(6-chloropyrazin-3-yl)-diazabicyclo[3.2.1]octane dihydrochloride monohydrate), FUB 181 (CAS No. 152029-80-6), RJR 2557, WSU 2088, LVV-haemorphin-7, M40 (i.e., galanin[1-12]-Pro₃-(Ala-Leu)₂-Ala-NH₂), SIB 1757, SKF 74652 (i.e., [5-chloro-2-(4-methoxypheny)-3-benzofuranyl][4-[3-(dimethyl amino)-propoxy]phenyl]methanone), CGP 71982, SCH 57790 (i.e., 4-cyclohexyl-α-[4-[[4-methoxypheny]sulfinyl]phenyl]-1-piperazineacetonitrile), Putrescine-D-YiAβ11, DU 14 (i.e., p-O-(sulfamoyl)-N-tetradecanoyltyramine), CLZ 4, SL 340026, PPRT 424, ciproxifan, UR 1827 (i.e., 2-(1-benzylpiperidin-4-yl)-1-[4-(5-methylpyrimidin-4-ylamino)phenyl]-1-ethanone), caproctamine, TGS 20 (i.e., L-pyroglutamyl-D-alanine amide), PG 9 (i.e., α-tropanyl 2-[(4-bromo)phenyl]propionate),
TEI 3356 (i.e., (16S)-15-deoxy-16-hydroxy-16-methyl-9-(O)-methano-Δ^{6(9α)}-prostaglandin I1), LY 392098 (i.e., thiophene, 3-[(2-methylethy-2)sulfonylaminopropyl-2]phenyl-4-yl-), PG 1000, DM 232, NEPP 11 (i.e., 12-iso-15-deoxy-18-(4-methyl)phenyl-13,14-dihydro-Δ7-prostaglandin A1 methyl ester), VA 100 (i.e., (2,3-dihydro-2-[[(4-fluorobenzoyl)amino]ethyl]-1-methyl-5-phenyl-1H-1,4-benzodiazepine), VA 101 (i.e., (2,3-dihydro-2-[[(2-thienylcarbonyl)amino]ethyl]-1-methyl-5-phenyl-1H-1,4-benzodiazepine)), NC 111585 (i.e., (3S)-1,3-bis-[3-[(3-azabicyclo[2.2.2]octanyl)-1,2,5-thiadiazol-4-yloxy]-1-propyn-1-yl]benzene, 2L-(+)-tartrate), IN 201, imoproxifan, kanokodiol, picroside I, picroside II, DM 235 (i.e., 1-(4-benzoylpiperazin-1-yl)propan-1-one), monoclonal antibody 10D5, JLK2, JLK6, JLK7, DAPT (i.e., N—[N-(3,5-difluorophenacetyl)-L-alanyl]-S-phenylglycine t-butyl ester), huperzine X, SGS 111 (i.e., (S)-ethyl 2-[1-(2-phenylacetyl)pyrrolidine-2-carboxamide]acetate), NP 7557, C 9136, C 7617, R 1485, rofecoxib, velnacrine, montirelin, lazabemide, ORG 2766 (CAS No. 50913-82-1), sabeluzole, adafenoxate, CAS No. 9061-61-4, ipidacrine, bemesetron, idazoxan, linopirdine, selfotel, suritozole, milameline, xanomeline, TJ960, fasoracetam, eptastigmine, ensaculin, zanapezil, posatirelin, zacopride, RS 86 (CAS No. 3576-73-6), ORG 5667 (CAS No. 37552-33-3), RX 77368 (CAS No. 76820-40-1), BMS 181168 (CAS No. 123259-91-6), BY 1949 (CAS No. 90158-59-1), AWD 5239 (CAS No. 109002-93-9), YM 796 (171252-79-2), aloracetam, CI 933 (CAS No. 91829-95-7), ST 793 (CAS No. 99306-37-3), cebaracetam, zifrosilone, talsaclidine, alvameline, JTP 2942 (148152-77-6), OPC 14117 (CAS No. 103233-65-4), elziverine, AP 521 (i.e., N-(1,3-benzodioxol-5-ylmethyl)-1,2,3,4-tetrahydro[1]benzothieno[2,3-c]pyridine-3(R)-carboxamide hydrochloride), S 8510 (CAS No. 151466-23-8), JTP 4819 (CAS No. 162203-65-8), icopezil, SC 110, FK 960 (CAS No. 133920-70-4), DMP 543 (CAS No. 160588-45-4), ganstigmine, CI 1017 (i.e., (R)-(−)-(Z)-1-azabicyclo[2.2.1]heptan-3-one, O-(3-(3′-methoxypheny)-2-propionyl)-oxime maleate), T 82 (i.e., 2-[2-(1-benzylpiperidin-4-yl)ethyl]-2,3-dihydro-9-methoxy-1H-pyrrolo[3,4-b]quinolin-1-one hemifumarate), NGD 971, vaccine of aspartyl-alanyl-glutamyl-phenylalanyl-arginyl-histidyl-aspartyl-seryl-glycyltyrosyl-glutamyl-valyl-histidyl-histidyl-glutaminyl-lysyl-leucyl-valyl-phenylalanyl-phenylalanyl-alanyl-glutamyl-aspartyl-valyl-glycyl-seryl- asparaginyl-lysyl- glycyl-alanyl-isoleucyl-isoleucyl-glycylleucyl-methionyl- valyl-glycyl-glycyl-valyl-valyl-isoleucyl-alanine, PBT 1 (CAS No. 130-26-7), TCH 346, FK 962 (i.e., N-(1-acetylpiperidin-4-yl)-4-fluorobenzamide), voxergolide, KW 6055 (CAS No. 63233-46-5), thiopilocarpine, ZK 93426 (CAS No. 89592-45-0), SDZ NVI 085 (CAS No. 104195-17-7), CI 1002 (CAS No. 149028-28-4),
Z 321 (CAS No. 130849-58-0), mirisetron, CHF 2060 (i.e., N-heptylcarbamic acid 2,4a,9-trimethyl-2,3,4,4a,9,9a-hexahydro-1,2-oxazino[6,5-b]indol-6-yl ester-L-tartrate), gedocarnil, terbequinil, HOE 065 (CAS No. 123060-44-6), SL 650102, GR 253035, ALE 26015, SB 271046 (i.e., 5-chloro-N-(4-methoxy-3-piperazin-1-yl-phenyl)-3-methyl-2-benzothiophene sulfonamide), iAβ5, SCH 211803 (i.e., 3-chlorophenyl[4-[1-[1-(2-amino-3-methylbenzoyl)-4-piperidinyl]-4-piperidinylmethyl]phenyl]sulfone), EVT 301, α-linolenic acid/linoleic acid, Kamikihi-to, siagoside, FG 7142 (CAS No. 78538-74-6), RU 47067 (CAS No. 111711-92-3), RU 35963 (CAS No. 139886-03-6), FG 7080 (CAS No. 100332-18-1), E 2030 (CAS No. 142007-70-3), transforming growth factor β-1, A 72055 (i.e., 2′,1-dimethylspiro[piperidine-4,5′-oxazolidine]-3′-carboxyaldehyde), NS 626, dimiracetam, GT 3001, GT 2501, GT 2342, GT 2016 (CAS No. 152241-24-2), ORG 20091 (CAS No. 141545-50-8), BCE 001 (CAS No. 95678-81-2), CGP 35348 (CAS No. 123690-79-9), WAY 100635 (CAS No. 146714-97-8), E 4804 (CAS No. 162559-34-4), LIGA 20 (CAS No. 126586-85-4), NG 121 (i.e., 2-[4,8-dimethyl-3(E),7(E)-nonadienyl]-3,5-dihydroxy-2-methyl-3,4,7,9-tetrahydro-2H-fluoro[3,4-h]-1-benzopyran-7-one), MF 247 (i.e., N-[10-(diethylamino)decyl]carbamic acid (3aS,8aR)-1,3a,8-trimethyl-1,2,3,3a,8,8a-hexahydropyrrolo[2,3-b]indol-5-yl ester), JTP 3399 (i.e., N-benzyl-2 (S)-[2(S)-(phenoxyacetyl) pyrrolidin-1-ylcarbonyl]pyrrolidine-1-carboxamide), KF 17329, thioperamide, F 3796 (i.e., 1-[2-(1-benzylpiperidin-4-yl)ethyl]-3-[3, 4-(methylene-dioxy)benzoyl]thiourea), GT 4001, GT 4002, FPL 14995 (CAS No. 123319-03-9), RU 34332 (CAS No. 137157-58-5), SR 96777A (CAS No. 115767-94-7), SIB T1980, NS 649 (CAS No. 146828-02-6), PD 142505 (CAS No. 149929-08-8), GYKI 52466 (CAS No. 102771-26-6), RO 246173 (CAS No. 159723-57-6), SCH 50911 (CAS No. 160415-07-6), Z 4105 (CAS No. 119737-52-9), RS 67333 (CAS No. 168986-60-5), NS 1546, ZM 241385 (CAS No. 139180-30-6), RO 249975 (i.e., [1S,3S(2'S),5R]-3-(1-benzyl-5-oxopyrrolidin-2-ylmethyl)-5-(1H-imidazol-5-ylmethyl)cyclohexane-1-acetamide), AF 185 (i.e., 8-methyl-3-(2-propynyl)-1,3,8-triazaspiro [4,5]decane-2,4-dione), CEP 427, CX 423, CX 438, CX 480, CDP-ethanolamine, GT 4003, GT 4011, GT 5011, MS 430 (CAS No. 122113-44-4), MBF 379 (i.e., [3,3-bis(hydroxymethyl)-8-hydroxy-3,4-dihydro-2H-1,4-benzoxazin-5-yl][3′,5′-dihydroxy-4′-(2-oxo-2-phenylethoxy)phenyl]methanone), NGD 187 (CAS No. 163565-48-8), DUP 856, MR 3066, MF 8615 (i.e., 5-amino-6-chloro-4-hydroxy-3,4-dihydro-1H-thiopyrano-[3,4-b]quinoline), himbacine, ABS 300, RJR 2403 (CAS No. 538-79-4), MF 268 (CAS No. 174721-00-7), RO 465934 (i.e., N,N-dimethylcarbamic acid 3-(2-cyclohexyl)-2,3,3a,4,5,9b-hexahydro-1H-benzo[e]indol-6-yl ester), NS 393, RGH 2716 (CAS No. 134069-68-4),
WIN 678702 (12,12-bis(3-furyl)-6,11-dihydro-6,11-ethanobenzo[b]quinolizinium chloride), RS 66252 (i.e., 1-butyl-2-[(2′-(2H-tetrazol-5-yl)-biphenyl-4-yl)methyl]-1H-indole-3-carboxylic acid), AIT 034 (CAS No. 138117-48-3), NG 012 (CAS No. 131774-53-3), PD 142012 (CAS No. 5778-84-7), GT 4054, GT 4077, GT 4035, P 26 (CAS No. 152191-74-7), RGH 5279 (i.e., (−)-(13aR,13bS)-13a-ethyl-2,3,5,6,13a,13b-hexahydro-1H-indolo[3,2,1-de]pyrido[3,2,1-ij][1,5]naphthyridine-12-carboxylic acid 2-acetoxyethyl ester), AIT 083, CeNeS, estradiol (i.e., 1,3,5(10)-estratriene-3,17β-diol), WAY 132983 ((3R,4R)-3-(3-hexasulfanylpyrazin-2-yloxy)-1-azabicyclo[2.2.1]heptane hydrochloride), ABS 205, ABS 401, SX 3507 (i.e., 3-(3-propyl-1,2,4-oxadiazol-5-yl)quinoxalin-2(1H)-one), ARR 17779 (i.e., (−)-spiro[1-azabicyclo[2.2.2]octaene-3,5-oxazolidine]-2-one), XE 991 i.e., 10,10-bis(4-pyridylmethyl)anthracen-10 (9H)-one), phenethylnorcymserine, RO 657199, RJR 1781 (i.e., R(+)-2-(3-pyridyl)-1-azabicyclo[2.2.2.]octane), RJR 1782 (i.e., S(−)-2-(3-pyridyl)-1-azabicyclo[2.2.2.]octane), gilatide, tolserine, TC 2559 (i.e., (E)-N-methyl-4-[3-(5-ethoxypyridine)yl]-3-butene-1-amine), ER 127528 (i.e., 1-(3-fluorobenzyl)-4-[(2-fluoro-5,6-dimethoxy-1-indanon-2-yl)methyl]piperidine hydrochloride), thiatolserine, targacept, axonyx, cymserine, thiacymserine, monoclonal antibody 266, Apan-CH, DP 103, SPI 339 (i.e., 4-[3-(4-oxo-4,5,6,7-tetrahydroindol-1-yl)propionylamino]benzoic acid ethyl ester), S 37245 (i.e., 4-(1,4-benzodioxan-5-yl)-1-[3 (S)-hydroxy-5-nitro-indan-2-yl]-piperazine), LLG 88, AZD 2858, trometamol, AN 240, NG 002 (i.e., 5-hydroxy-5-(2-hydroxy-1-methylethyl)-4-methoxyfuran-2(5H)-one), UCB 29427 (i.e., 2-cyclopropyl-4-(cyclopropylamino)-6-(morpholino)-1,3,5-triazine), TRH-SR, RO 401641 (CAS No. 122199-02-4), MPV 1743AIII (CAS No. 150586-64-4), IDRA 21 (CAS No. 22503-72-6), CEP 431, ACPD (CAS No. 67684-64-4), CT 3577 (i.e., 3,7-dimethyl-1-[11-(3,4,5-trimethoxybenzylamino)-11-oxoundecyl]xanthine), CT 2583, NXD 9062, desferri-nordanoxamine, DP b99, PBT 1, T 817MA, Alfatradiol (CAS No. 57-91-0), AL 108, SL 650102, RS 67333 (CAS No. 168986-60-5), RS 17017, SGS 518, SYN 114, SB 271046, RO 657199, PRX 07034, Suritozole (CAS No. 110623-33-19), Terbequinil (CAS No. 113079-82-6), FG 7142 (CAS No. 78538-74-6), RU 34332 (CAS No. 137157-58-5), SX 3507, RO 153505 (CAS No. 78771-13-8), RU 33965 (CAS No. 122321-05-5), S 8510 (CAS No. 151466-23-8), Sabeluzole (CAS No. 104383-17-7), Cerebrocrast (CAS No. 118790-71-9), NS 626, NS 649 (CAS No. 146828-02-6), U 92032 (CAS No. 142223-92-5), MEM 1003, U 92798, RGH 2716 (CAS No. 134069-68-4), Safinamide (CAS No. 133865-89-1), AZD 0328, MEM 63908, ABT 418 (CAS No. 147402-53-7), ARR 17779, RJR 2403 (CAS No. 538-79-4), TC 2559, A 82695 (CAS No. 147388-86-1), A 84543, A 98284, DBO 83, RJR 2557, SIB 1765F (CAS No. 179120-52-6), GTS 21 (CAS No. 156223-05-1), MEM 3454, SIB 1553A, EVP 6124, SSR 180711,
ABT 089 (CAS No. 161417-03-4), ABT 107, ABT 560, TC 5619, TAK 070, N-[(1S,2R)-3-(3,5-difluorophenyl)-1-hydroxy-1-[(5S,6R)-5-methyl-6-(neopentyloxy)morpholin-3-yl]propan-2-yl]acetamide hydrochloride, 6-fluoro-5-(2-fluoro-5-methylphenyl)-3,4-dihydropyridine, 2-amino-6-[2-(3′-methoxybiphenyl-3-yl)ethyl]-3,6-dimethyl-5,6-hydroxypyrimidin-4 (3H)-one, AZD 1080, ARA 014418, XD 4241, Z 321 (CAS No. 130849-58-0), ONO 1603 (CAS No. 114668-76-7), JTP 3399, Eurystatin A (CAS No. 137563-63-4), Eurystatin B (CAS No. 137563-64-5), P 128 (CAS No. 157716-52-4), Y 29794 (CAS No. 129184-48-1), ZTTA 1, JTP 4819 (CAS No. 162203-65-8), monoclonal antibody 266, duloxetine, escitalopram oxalate, fluoxetine, fluvoxamine maleate, paroxetine, sertraline, dapoxetine, desvenlafaxine, sibutramine, nefazodone, milnacipran, desipramine, duloxetine, and bicifadine.

The composition of the present invention may be provided, if desired, in the form of a kit that comprises a container, such as a pack or a dispenser, capable of containing one or more unit dosage forms containing an active ingredient.

In the present invention, different pharmaceutical compositions may also be combined in the form of a kit. The kit may comprise two or more kinds of different pharmaceutical compositions. For example, the kit comprises the compound of the present invention and one or more compounds known to be useful for the treatment or prevention of AD, and/or the compound of the present invention and a compound that exerts medicinal benefits in the treatment of a disease other than AD. The kit usually comprises a container, such as a divided bottle or a divided foil packet, for separately containing the different compositions, but the different compositions may also be contained in a single, undivided container. The form of a kit is particularly advantageous when different components are preferably administered in different dosage forms (e.g., oral and parenteral), when different components are administered at different dosage intervals, or when titration of the individual components of the combination is desired by the prescribing physician.

The pack may be a blister pack, for example, that may comprise a metal foil or a plastics foil, for example. Blister packs are well known in the packaging industry and are widely used for the packaging of pharmaceutical unit dosage forms (tablets, capsules, and the like). In general, a blister pack preferably consists of a sheet of a relatively stiff material covered with a foil of a transparent plastic material. During the packaging process, cavities are formed in the plastic foil. The cavities have a size and a shape suitable for each capsule or the like to be packed. Next, the capsules or the like are placed in the cavities, and the plastic foil is sealed at the face opposite from the face on which the cavities have been formed with use of a sheet of relatively stiff material. As a result, the capsules or the like are sealed in the cavities between the plastic foil and the sheet. Preferably, the strength of the sheet is such that, when a pressure is manually applied to a cavity, the sheet can be broken at the site of the cavity to form an opening that allows the capsule or the like to be removed from the blister pack. The tablet or capsule can be removed via the opening.

A package insert for administration, a product insert, etc. can be attached to the pack or the dispenser. The pack, the dispenser and other containers used can be adapted to the notifications issued by the government agency or the authorities that regulate medicinal production, use, or sale.

In an embodiment of the present invention, the oral drug may be a drug for extending axons and/or a drug for repairing degenerated axons. The oral drug preferably has the mechanism of action of diosgenin or the like and exhibits the effects of extending axons and/or repairing degenerated axons. In this embodiment, the dosage and the subject of administration of the drug for extending axons and/or the drug for repairing degenerated axons may be as defined above.

The effects of extending axons or repairing degenerated axons of a compound can be evaluated by a known or conventional method commonly used in the art or an equivalent method thereof. Specifically, the effects can be determined as follows. Mice are anesthetized and transcardially perfused with cold physiological saline. The brains are carefully removed from the skull in a conventional manner, immediately immersed in 10 to 30% (w/v) sucrose-PBS and stored at −80° C. The brains are cut in 20-μm successive coronal slices every 100 μm in the parietal area (bregma 1.4-2 mm) sections using a cryostat (CM3050S, Leica, Heidelberg, Germany). The slices are fixed with 4% (w/v) paraformaldehyde/(0.1 mol/L) phosphate buffer and stained with a polyclonal antibody against Aβ (1-40/42) (1:300) (Chemicon, Temecula, Calif., USA) and a monoclonal antibody against pNF-H (1:500) (Covance, Emeryville, Calif., USA) at 4° C. for 20 hours. Alexa Fluor 488-conjugated goat anti-mouse IgG (1:300) and Alexa Fluor 568-conjugated goat anti-rabbit antibody (1:300) are used as secondary antibodies (Molecular Probes, Eugene, Oreg., USA). The fluorescent images for axons and Aβ (1-40/42) are captured using a fluorescent microscope (BX61) at 324 μm×430 μm. Three successive brain slices of the frontal cortex and five successive slices of the hippocampus are captured from a mouse for quantification. Extracellular amyloid plaques are determined by the size (greater than 50 μm in width), and the area of amyloid plaques is measured using the image analyzing software ImageJ (http://rsbweb.nih.gov/ij). The extension of axons is evaluated by measuring the length of pNF-H-positive fiber axons using Neurocyte (Kurabo, Osaka) or Metamorph (Molecular Devices, Sunnyvale, Calif., US). The repair of degenerated axons is evaluated by determining the area of pNF-H-positive bulb axons localized in the area of amyloid plaques using ImageJ.

More specific methods for evaluating the extension or repair of axons can be performed by referring to, for example, Tohda C, Urano T, Umezaki M, Nemere I, Kuboyama T, Diosgenin is an exogenous activator of 1,25D₃-MARRS/Pdia3/ERp57 and improves Alzheimer's disease pathologies in 5XFAD mice., Sci. Rep., 2, 535; DOI:10.1038/srep00535 (2012).

Another embodiment of the present invention relates to a food or drink, a feed, a food additive, a feed additive, or the like, each comprising the oral drug of the present invention.

Hereinafter, the food or drink of the present invention will be described below.

To the food or drink of the present invention, one or more kinds of food additives generally used in foods or drinks may be added, and examples thereof include a sweetener, a colorant, a preservative, a thickener, an antioxidant, a color improver, a decolorant, an antifungal agent, a gum base, a bittering agent, an enzyme, a brightener, an acidulant, a seasoning, an emulsifier, a fortifier, an agent for production, a flavor, a spice extract, etc. The food or drink of the present invention includes a health food, a functional food, a food for specified health use, and a food for babies, toddlers, pregnant or nursing mothers, the elderly, or the sick.

The form of the food or drink of the present invention is not particularly limited. Specific examples thereof include so-called dietary supplements, such as a tablet, a capsule, a granule, a powder, and a health drink. Other examples include drinks, such as tea drink, refreshing drink, soda, nutritional drink, fruit juice, and lactic acid drink; noodles, such as buckwheat noodle, wheat noodle, Chinese noodle, and instant noodle; sweets and bakery products, such as drop, candy, gum, chocolate, snack, biscuit, jelly, jam, cream, baked goods, and bread; fishery or livestock products, such as fish sausage, ham, and sausage; dairy, such as processed milk and fermented milk; fats, oils, and processed foods thereof, such as salad oil, oil for deep frying, margarine, mayonnaise, shortening, whipped cream, and dressing; seasonings, such as sauce and dipping sauce; retort pouch foods, such as curry, stew, rice-bowl cuisine, porridge, and rice soup; and frozen desserts, such as ice cream, sherbet, and shaved ice.

The intake of the food or drink of the present invention is not particularly limited and may be appropriately set depending on the form of the food or drink, the age, sex, conditions, etc. of a subject who is to take the food or drink, and other conditions.

Another embodiment of the present invention relates to a method for reducing amyloid plaques, tau deposition, tau precipitate, PHF-tau, or neurofibrillary tangles, the method comprising administering the oral drug of the present invention to a subject. In this embodiment, the subject of administration, the dosage, etc. may be the same as above.

Another embodiment of the present invention relates to a method for suppressing amyloid β (Aβ) (1-42)-induced axonal atrophy, the method comprising administering the oral drug of the present invention to a subject. In this embodiment, the subject of administration, the dosage, etc. may be the same as above.

Another embodiment of the present invention relates to a method for activating a signaling pathway through stimulation of 1,25D₃-MARRS, the method comprising administering the oral drug of the present invention to a subject. In this embodiment, the subject of administration, the dosage, etc. may be the same as above.

A further embodiment of the present invention relates to a method for enhancing or improving normal memory, the method comprising administering the oral drug of the present invention to a subject. The “normal memory” includes “the memory of a subject without a disease in which amyloid plaques, tau deposition, tau precipitate, PHF-tau, or neurofibrillary tangles, or Aβ (1-42)-induced axonal atrophy is observed”. In this embodiment, the subject of administration, the dosage, etc. may be the same as above.

### Diosgenin Derivative and Application Thereof

Another embodiment of the present invention includes a novel diosgenin derivative. The diosgenin derivative is exemplified by the novel diosgenin derivative disclosed herein for the first time (for example, a compound represented by formula (III)), which is encompassed in the compound of formula (I-1) or a (pharmaceutically acceptable) salt thereof.

The present invention also includes a prophylactic or therapeutic drug for a disease associated with axonal dysfunction, the drug comprising a diosgenin derivative (for example, a compound represented by formula (I-1) or a salt thereof).

The disease associated with axonal dysfunction is exemplified by the above diseases such as spinal cord injury, brain contusion, Alzheimer's disease (AD), Parkinson's disease, and dementia. Of these diseases, AD or spinal cord injury is particularly preferred.

The present invention further includes a drug for extending axons or a drug for repairing degenerated axons, each drug comprising a diosgenin derivative.

The present invention further include a medicament (or a pharmaceutical composition) comprising a diosgenin derivative.

The present invention further includes a functional health food comprising a diosgenin derivative.

The dosage form of the diosgenin derivative in such various applications (the prophylactic drug, the therapeutic drug, the drug for extending axons, the drug for repairing axons, the functional health food, etc.) is not limited to the particular oral dosage form as a suspension or solution in an oil or fat, as long as the dosage form comprises the diosgenin derivative. Various dosage forms of the diosgenin derivative are possible.

The dosage form include a tablet, a suspension, a powder, a fine granule, a granule, a dry syrup, a coated tablet, an orally disintegrating tablet, a chewable tablet, a capsule, a soft capsule, a syrup, an oral solution, a troche, a jelly, a inhalation, a suppository, an injection, an ointment, an eye drop, an eye ointment, a nasal drop, an ear drop, a cataplasm, a lotion, a liquid for external use, a spray, an aerosol for external use, a cream, a gel, a tape, a buccal tablet, a sublingual tablet, a vaginal suppository, a vaginal tablet, a rectal soft capsule, etc. In the formulation, conventionally used additives, such as an excipient, a binder, a disintegrant, a coating agent, a lubricant, a colorant, and an odor corrective; and as needed, a stabilizer, an emulsifier, an absorption promoter, a surfactant, a pH adjuster, an antiseptic, an antioxidant, etc. may be used. The formulation can be performed in the usual manner using components usually used as raw materials of a pharmaceutical composition.

The administration route is not particularly limited, and may be oral or parenteral. Examples of the parenteral administration include, for example, rectal, nasal, intrapulmonary, and injection administration (for example, intravenous, intraspinal, epidural, intramuscular, subcutaneous, intraperitoneal, intraarterial, intraarticular, intracardiac, intracapsular, intracutaneous, intralesional, intraocular, intrapleural, subarachnoid, intrauterine, and intraventricular administration), etc.

The aspects (such as the dosage, the subject of administration, etc.) other than being an oral drug as described above are the same as above.

### EXAMPLES

The present invention will be described in further detail with reference to Experimental Examples and Examples, but the present invention is not limited thereto. Various modifications are possible within the technical idea of the present invention by a person who has ordinary knowledge in the art.

### Statistical Analysis

The results obtained in the Examples below were evaluated by the following statistical tests.

One-way analysis of variance (one-way ANOVA), post hoc Dunnett's test, and paired t-test were performed using Graphpad Prism 5 (Graphpad Software, La Jolla, Calif., USA). Statistical differences were considered significant when the p value was <0.05. The means of the data are presented together with the SE.

### Animals

### (1) Normal Mice

ddY Mice were obtained from Japan SLC (Hamamatsu, Japan) In the Examples below, 6-week-old male ddY mice were used as normal mice. All mice were housed with free access to food and water and were kept in a controlled environment at 22±2° C., 50±5% humidity, and 12-h light/dark cycle starting at 7:00 am.

### (2) AD Model Mice

Transgenic mice (5XFAD) as an animal model of AD were obtained from the Jackson Laboratory (Bar Harbor, Me., USA). The 5XFAD mice overexpress human APP695 cDNA having Swedish mutation (K670N and M671L), Florida mutation (I716V) and London mutation (V717I) and human PS1 cDNA (M146L and L286V) under the transcriptional control of the neuron-specific mouse Thy-1 promoter (Oakley, H. et al., J Neurosci, 26, 10129-10140, 2006). They were maintained by crossing hemizygous transgenic mice with B6/SJL F1 breeders.

In the Examples below, 24- to 27-week-old male and female 5XFAD mice or 28- to 31-week-old female 5XFAD mice were used as AD model mice. All mice were housed with free access to food and water and were kept in a controlled environment (22±2° C., 50±5% humidity, 12-h light/dark cycle starting at 7:00 am).

### Measurement of Spontaneous Motor Activity

In Reference Test 1, the spontaneous motor activity was measured as follows.

For each mouse to be tested, the path during the 10-minute habituation in an open-field box was tracked using a digital camera system. The distance each mouse traveled during the 10 minutes was analyzed as locomotion activity with EthoVision 3.0 (Noldus, Wageningen, The Netherlands).

### Object Recognition Memory Test

In the Examples below, an object recognition memory test was performed as follows.

The next day of the measurement of spontaneous motor activity, an object recognition memory test was performed as described in Joyashiki, E. et al., Int J Neurosci, 121, 181-190, 2011 and Tohda, C. et al., Int J Neurosci, 121, 641-648, 2011, reported by the inventors and others. Testing was carried out in a dimly illuminated room (about 100 lx). An appropriate time interval between a training session and a test session was set based on the results of a preliminary test using another group of mice. The object recognition memory test utilizes animals' habit of showing interest in a novel object. The inner walls of the open-field box for the test had no marks. In the training session, two identical objects were placed in the field, and mice were allowed to explore freely for 10 minutes. In the test session, the location of the objects was not changed, but one of the objects was replaced with a novel object, and the mice were allowed to explore freely for 10 minutes. The increase of the number of times a mouse preferentially explored the novel object after replacement was used as an index of the object memory. That is, the test session determines whether a mouse remembers the previous exposure to an object in the training session. In the Examples below, the ratio (%) of the number of times a mouse explored the novel object to the total exploration time was calculated as a preference index.

### Object Location Memory Test

In the Examples below, an object location memory test was performed as follows.

Testing was carried out in a dimly illuminated room (about 100 lx). An appropriate time interval between a training session and a test session was set based on the results of a preliminary test using another group of mice. The object location memory test utilizes animals' habit of showing interest in a novel object. The two opposite walls of a four-walled open-field box for the test had wall coverings with a distinct pattern. In the training session, two identical objects that were shown to mice for the first time were placed in the field, and the mice were allowed to explore freely for 10 minutes. In the test session, one of the objects was moved to a different location, and the mice were allowed to explore freely for 10 minutes. The increase of the number of times a mouse preferentially explored the identical but relocated object was used as an index of the location memory. That is, the test session determines whether a mouse remembers the previous exposure to an object in the training session. In the Examples below, the ratio (%) of the number of times a mouse explored the object after relocation to the total exploration time was calculated as a preference index. The test was performed by referring to Tohda C., Joyashiki E., Sominone enhances neurite outgrowth and spatial memory mediated by the neurotrophic factor receptor, RET. British Journal of Pharmacology (2009) 157, 1427-1440.

### Example 1

To 2.07 mg of diosgenin (Wako Pure Chemical Industries, Ltd.) was added 5 mL of sesame oil (KANEDA Co., Ltd.). The mixture was stirred with a microhomogenizer to give a uniform suspension. A 0.5 mL aliquot of the suspension was uniformly mixed with 49.5 mL of sesame oil to give a suspension containing diosgenin at 0.00414 mg/mL in the sesame oil (Example Product 1). Example Product 1 was orally administered to the AD model mice (5XFAD, male and female, 24 to 27 weeks old) once a day at a diosgenin dosage of 0.1 μmol/kg·day per unit weight of the animal. The administration period was 20 days. The mice were then subjected to the object recognition memory test. The training session was performed on the next day of the final administration. The interval between the training session and the test session was 1 hour.

### Example 2

The suspension preparation, the administration and the memory test were performed in the same manner as in Example 1 except that the dosage of diosgenin was 10 μmol/kg·day per unit weight of the animal.

### Example 3

The suspension preparation, the administration and the memory test were performed in the same manner as in Example 1 except that Example Product 3 prepared by replacing the sesame oil with olive oil (KANEDA Co., Ltd.) was used.

### Example 4

The suspension preparation, the administration and the memory test were performed in the same manner as in Example 1 except that Example Product 4 prepared by replacing the sesame oil with soybean oil (KANEDA Co., Ltd.) was used.

### Comparative Example 1

The administration and the memory test were performed in the same manner as in Example 1 except that sesame oil alone was used instead of Example Product 1.

### Control

The administration and the memory test were performed in the same manner as in Comparative Example 1 except that wild-type mice (24 to 27 weeks old) were used instead of the AD model mice.

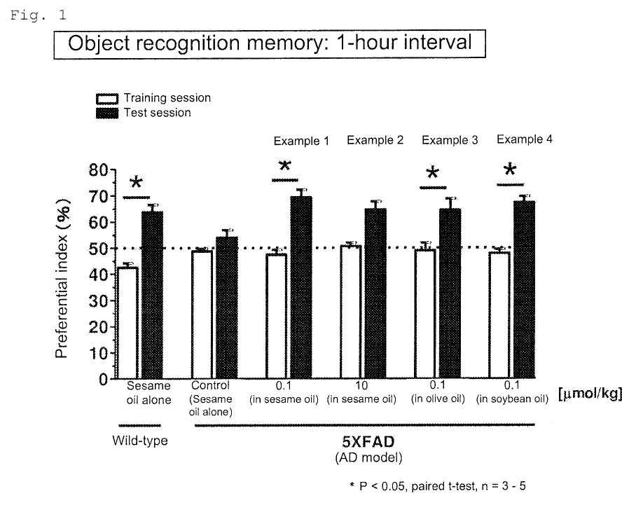

FIG. 1 shows the results.

The memory deficits of the mice in Examples 1 to 4 were improved to be comparable with the wild-type mice as the control.

### Example 5

To 1.30 mg of (3β,25R)-3-(2-aminoethanoyloxy)-spirost-5-ene hydrochloride (a synthesized product, hereinafter abbreviated as Dios-G) was added 2.544 mL of sesame oil (KANEDA Co., Ltd.). The mixture was stirred with a microhomogenizer to give a uniform suspension. A 0.5 mL aliquot of the suspension was uniformly mixed with 49.5 mL of sesame oil to give a suspension containing Dios-G at 0.005081 mg/mL in the sesame oil (Example Product 5).

Example Product 5 was orally administered to the AD model mice (5XFAD, female, 28 to 31 weeks old) once a day at a Dios-G dosage of 0.1 μmol/kg·day per unit weight of the animal. The administration period was 20 days. The mice were then subjected to the object recognition memory test. The training session was performed on the next day of the final administration. The interval between the training session and the test session was 1 hour.

### Example 6

The suspension preparation, the administration and the memory test were performed in the same manner as in Example 5 except that Example Product 6 prepared by replacing the synthesized product with (3β,25R)-3-fluorospirost-5-ene (a synthesized product, hereinafter abbreviated as Dios-F) was used instead of Example Product 5. Example Product 6 was prepared as follows. To 1.13 mg of Dios-F was added 2.712 mL of sesame oil, and the mixture was stirred to give a uniform suspension. A 0.5 mL aliquot of the suspension was uniformly mixed with 49.5 mL of sesame oil to give a suspension containing Dios-F at 0.004166 mg/mL in the sesame oil (Example Product 6)

### Comparative Example 2

The administration and the memory test were performed in the same manner as in Example 5 except that sesame oil alone was used instead of Example Product 5.

### Control

The administration and the memory test were performed in the same manner as in Comparative Example 2 except that wild-type mice (31 weeks old) were used instead of the AD model mice.

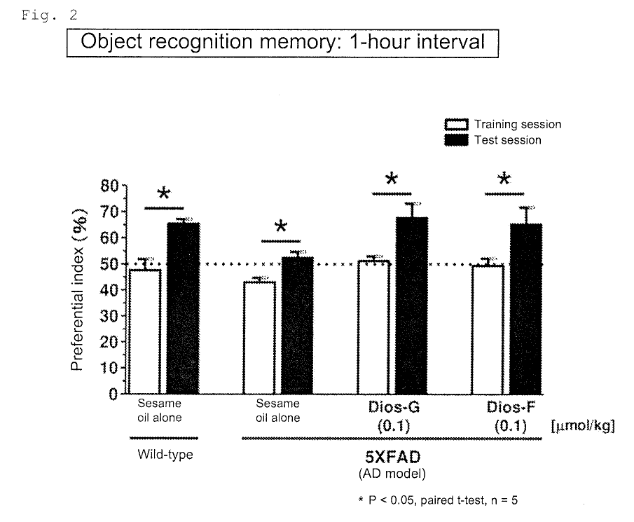

FIG. 2 shows the results.

The memory deficits of the mice in Examples 5 and 6 were improved to be comparable with the wild-type mice used as the control.

### Example 7

Dios-G was suspended in sesame oil in the same manner as in Example 5 to give a suspension (Example Product 7). Example Product 7 was orally administered to the AD model mice (5XFAD, female, 28 to 31 weeks old) once a day at a Dios-G dosage of 0.1 μmol/kg·day per unit weight of the animal. The administration period was 25 days. The mice were then subjected to the object recognition memory test. The training session was performed on the next day of the final administration. The interval between the training session and the test session was 24 hours.

### Example 8

The suspension preparation, the administration and the memory test were performed in the same manner as in Example 7 except that a suspension (Example Product 8) prepared by suspending Dios-F in sesame oil in the same manner as in Example 6 was used.

### Comparative Example 3

The administration and the memory test were performed in the same manner as in Example 7 except that sesame oil alone was used instead of Example Product 7.

### Control

The administration and the memory test were performed in the same manner as in Comparative Example 3 except that wild-type mice (31 weeks old) were used instead of the AD model mice.

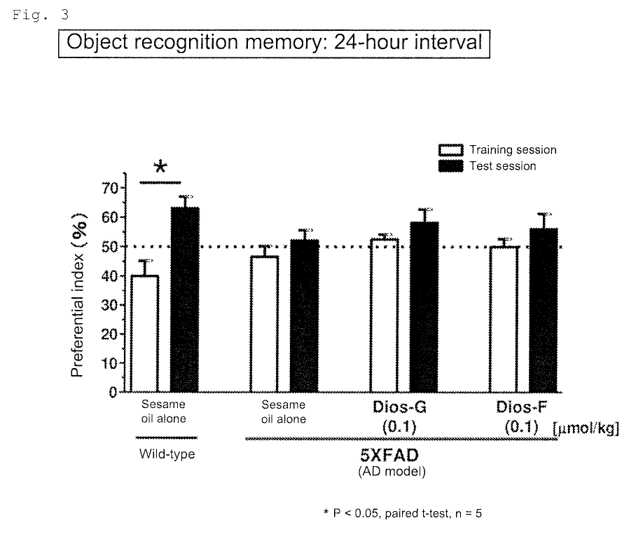

FIG. 3 shows the results.

In the object recognition memory tests with an extended interval of 24 hours, the memory deficits of the mice in Example 7 and 8 appeared to be improved.

### Example 9

Dios-F was suspended in sesame oil in the same manner as in Example 6 to give a suspension (Example Product 9). Example Product 9 was orally administered to the AD model mice (5XFAD, female, 28 to 31 weeks old) once a day at a Dios-F dosage of 0.1 μmol/kg·day per unit weight of the animal. The administration period was 22 days. The mice were then subjected to the object location memory test. The training session was performed on the next day of the final administration. The interval between the training session and the test session was 24 hours.

### Comparative Example 4

The administration and the memory test were performed in the same manner as in Example 9 except that sesame oil alone was used instead of Example Product 9.

### Control

The administration and the memory test were performed in the same manner as in Comparative Example 4 except that wild-type mice (31 weeks old) were used instead of the AD model mice.

The results indicated an improvement in the memory deficits of the mice in Example 9.

### Test Example: Evaluation of Hindlimb Motor Function Based on BMS and TMS

Tests were performed using spinal cord injury model mice to assess the effects of a diosgenin derivative on the hindlimb function of the mice.

### Spinal Cord Injury (SCI) Mice

Contusion injury was induced in 8-week-old female ddY mice (SLC) by the following procedure to give spinal cord injury (SCI) model mice. The ddY mice were maintained with free access to food and water in a controlled environment (22±2° C., 50±5% humidity, 12-h light/dark cycle starting at 7:00 am) Contusion injury was induced by exposing the lumbar vertebrae of each mouse in accordance with a conventional procedure, then placing the mouse in a stereotaxic apparatus (Narishige), and dropping a 6.5-g weight onto the first lumbar vertebrae (L1) from a height of 2 cm once. The mouse was then subjected to surgical procedures such as suture in a conventional manner. One hour after the induction of the contusion injury, the mice were randomly selected and divided into test groups and were subjected to the administration test described later in Example 10 and Comparative Example 5.

### Test Method

Each mouse after the administration test was moved to an open field (42 cm×48 cm×15 cm) and was observed for 5 minutes for the evaluation of hindlimb motor function. The locomotion behavior in the open field was evaluated using the Basso Mouse Scale (BMS) (see, for example, Engesser-Cesar C, Anderson A J, Basso D M et al., (2005), Voluntary wheel running improves recovery from a moderate spinal cord injury, J Neurotrauma 22: 157-171), which was a general standard for the evaluation of the hindlimb motor function of spinal cord injury model mice, and a 0-30-point Toyama Mouse Scale (TMS), which was a modified BMS established by the inventors for better test accuracy.

The new Toyama Mouse Scale (TMS), which was a modified BMS established by the inventors for better test accuracy, was used for the evaluation of the hindlimb function of SCI mice. The TMS score table is shown in Table 1. The numbers in the parentheses in the table indicate the scores. The scores for each item were determined and then summed for the evaluation.

TABLE 1

| Ankle movement | | | | | | | | Touchable area of the sole | | | | | | Hindlimb | | | |
|---|---|---|---|---|---|---|---|---|---|---|---|---|---|---|---|---|---|
| | | Mobile | | Movement in | | Toe | | At | | At | | Coor- | | movement at | | | |
| Frequency | | extent | | other joints | | movement | | resting | | stepping | | dination | | stepping | | Body support | |
| No | 0 | No | 0 | No | 0 | No | 0 | No | 0 | No | 0 | No | 0 | No | 0 | No | 0 |
| <50% | 1 | <50% | 1 | Yes | 1 | Yes | 1 | Partial sole | 1 | Partial | 1 | Yes | 1 | Rotative | 1 | Sometimes | 5 |
| | | | | | | | | touch | | sole touch | | | | | | support of hind | |
| | | | | | | | | | | | | | | | | body trunk | |
| ≧50% | 2 | ≧50% | 2 | | | | | Full sole | 2 | Full sole | 2 | | | Parallel | 2 | Always support | 10 |
| | | | | | | | | touch | | touch, | | | | | | of the body | |
| | | | | | | | | | | frequen- | | | | | | trunk, but | |
| | | | | | | | | | | cy <50% | | | | | | unstable weight | |
| | | | | | | | | | | | | | | | | support | |
| | | | | | | | | | | Full sole | 3 | | | | | Always support | 15 |
| | | | | | | | | | | touch, | | | | | | of the body | |
| | | | | | | | | | | frequen- | | | | | | trunk, and stable | |
| | | | | | | | | | | cy ≧50% | | | | | | weight support | |
| | | | | | | | | | | Full sole | 4 | | | | | | |
| | | | | | | | | | | touch in | | | | | | | |
| | | | | | | | | | | every step | | | | | | | |

### Example 10

Dios-F was suspended in sesame oil in the same manner as in Example 6 to give a suspension (Example Product 10). Example Product 10 was orally administered to the spinal cord injury mice once a day at a Dios-F dosage of 0.1 μmol/kg·day per unit weight of the animal. The first administration was performed one hour after the induction of the contusion injury, and the second administration was performed on the next day (one day after the induction). The administration period was 14 days. The mice were then subjected to the evaluation of the hindlimb motor function.

### Comparative Example 5

The administration and the evaluation were performed in the same manner as in Example 10 except that sesame oil alone was used instead of Example Product 10.

In Example 10, the number of the mice tested was 3, and therefore the number of the hindlimbs tested was 6 in total (n=6). In Comparative Example 5, the number of the mice tested was 6, and therefore the number of the hindlimbs tested was 12 in total (n=12).

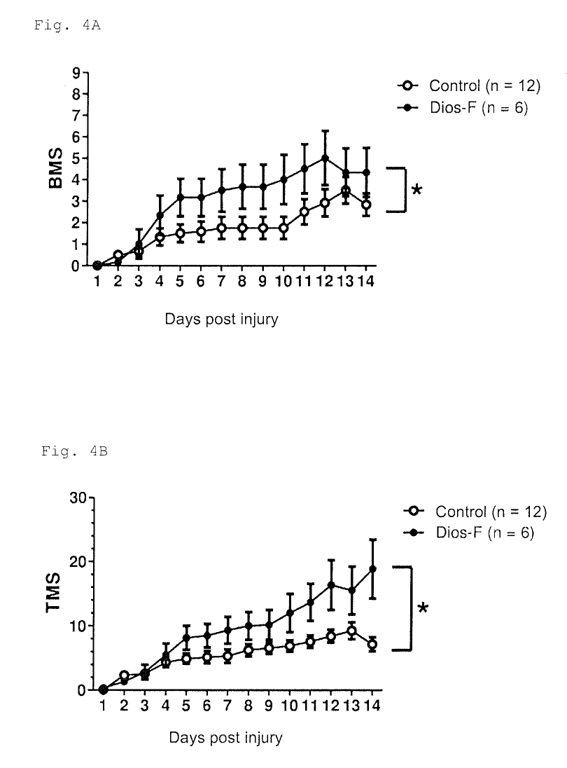

FIGS. 4A and 4B show the results. FIG. 4A shows the Basso Mouse Scale (BMS) scores, and FIG. 4B shows the Toyama Mouse Scale (TMS) scores. The hindlimb motor function of the mice with the oral administration of Dios-F (Example 10, the group indicated by the black circles in FIGS. 4A and 4B) was significantly improved as compared with that of the mice with administration of sesame oil alone as a control (Comparative Example 5, the group indicated by the white circles in FIGS. 4A and 4B).

### Reference Test 1: Assessment of Effects of Oral Administration of Diosgenin Derivatives Suspended in Edible Oil on Spontaneous Motor Activity and Body Weight Changes

Dios-G and Dios-F were separately suspended in sesame oil in the same manner as in Examples 5 and 6 to prepare suspensions. Each of the suspensions was orally administered to the AD model mice (5XFAD, female, 28 to 31 weeks old) once a day at a dosage of the diosgenin derivative (Dios-G or Dios-F) of 0.1 μmol/kg·day per unit weight of the animal. One hour after the administration on Day 20, the spontaneous motor activity was determined. The administration was then further continued. The total administration period was 25 days. During the administration period of 25 days, the body weight was determined every day.

As controls, sesame oil alone was administered to the AD model mice (5XFAD, female, 28 to 31 weeks old) and wild-type mice (31 weeks old). The spontaneous motor activity on Day 20 of the administration and the body weights were determined.

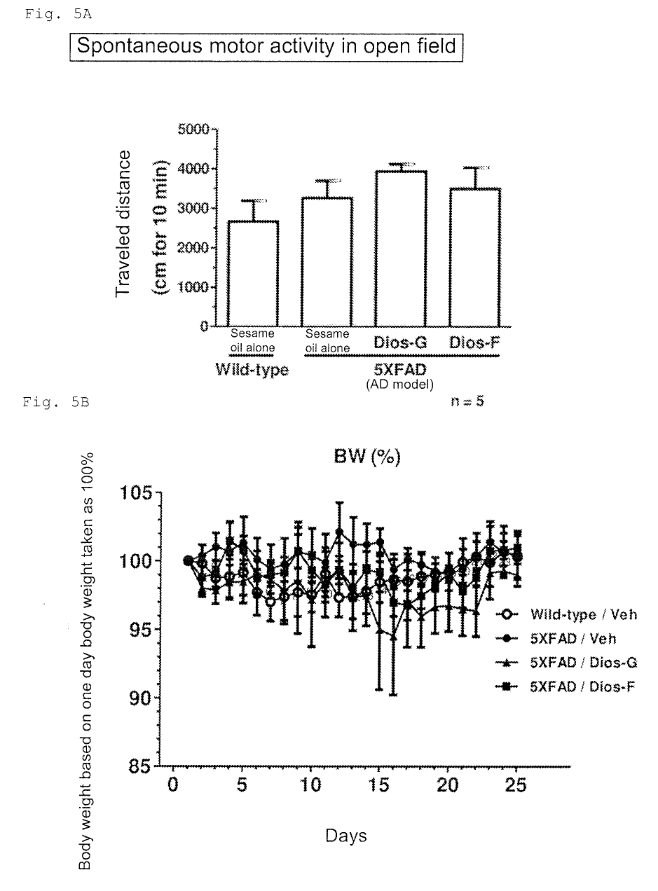

FIG. 5A shows the results of the spontaneous motor activity, and FIG. 5B shows the results of weight measurement.

No significant difference was observed in the spontaneous motor activity and the body weight among the AD model mice with the administration of the diosgenin derivatives, and the AD model mice and wild-type mice without the administration of the diosgenin derivatives.

### Reference Test 2: Oral Administration of Diosgenin Dissolved in an Aqueous Solvent Shows No Memory Enhancing Effect Reference Example 1

In 1.182 mL of ethanol was dissolved 4.9 mg of diosgenin to give a 10 mM diosgenin solution in ethanol. Separately, 2.3 g of glucose was dissolved in 46 mL of water to give a 5% aqueous glucose solution. A 1 mL aliquot of the 10 mM diosgenin solution in ethanol was added to 9 mL of the 5% aqueous glucose solution, and the mixture was mixed to give a diosgenin solution in an aqueous solvent (Reference Product 1). Reference Product 1 was orally administered to the normal mice (ddY, male, 6 weeks old) once a day at a diosgenin dosage of 10 μmol/kg·day per unit weight of the animal. The administration period was 5 days. The mice were then subjected to the object location memory test. The training session was performed on the next day of the final administration. The interval between the training session and the test session was 48 hours.

### Reference Example 2

The solution preparation, the administration and memory test were performed in the same manner as in Reference Example 1 except that a diosgenin solution in an aqueous solvent (Reference Product 2) prepared in the same manner as in Reference Example 1 was intraperitoneally administered.

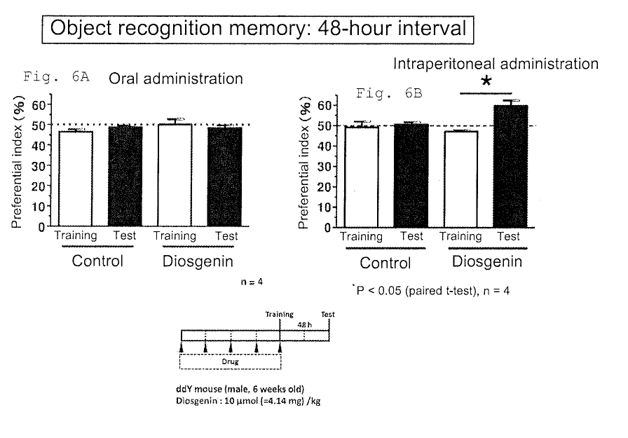

FIGS. 6A and 6B show the results. The mice with the oral administration of diosgenin dissolved in an aqueous solvent in Reference Example 1 showed no memory enhancing effect (FIG. 6A). In contrast, the mice with the intraperitoneal administration of diosgenin dissolved in an aqueous solvent in Reference Example 2 showed a significant memory enhancing effect.

### Reference Test 3: Assessment of Memory Enhancing Effects of Diosgenin at Low Dose on Normal Mice

### Reference Example 3

A diosgenin solution in an aqueous solvent (Reference Product 3) was prepared in the same manner as in Reference Example 1. Reference Product 3 was intraperitoneally administered to the normal mice (ddY, male, 6 weeks old) once a day at a diosgenin dosage of 0.1 μmol/kg·day per unit weight of the animal. The administration period was 7 days. The mice were then subjected to the object recognition memory test. The training session was performed on the next day of the final administration. The interval between the training session and the test session was 48 hours.

### Reference Example 4

The solution preparation, the administration and the memory test were performed in the same manner as in Reference Example 3 except that the dosage of diosgenin was 1 μmol/kg·day per unit weight of the animal.

### Reference Example 5

The solution preparation, the administration and the memory test were performed in the same manner as in Reference Example 3 except that the dosage of diosgenin was 10 μmol/kg·day per unit weight of the animal.

### Control

The administration and the memory test were performed in the same manner as in Reference Example 3 except that sesame oil alone was used instead of Reference Product 3.

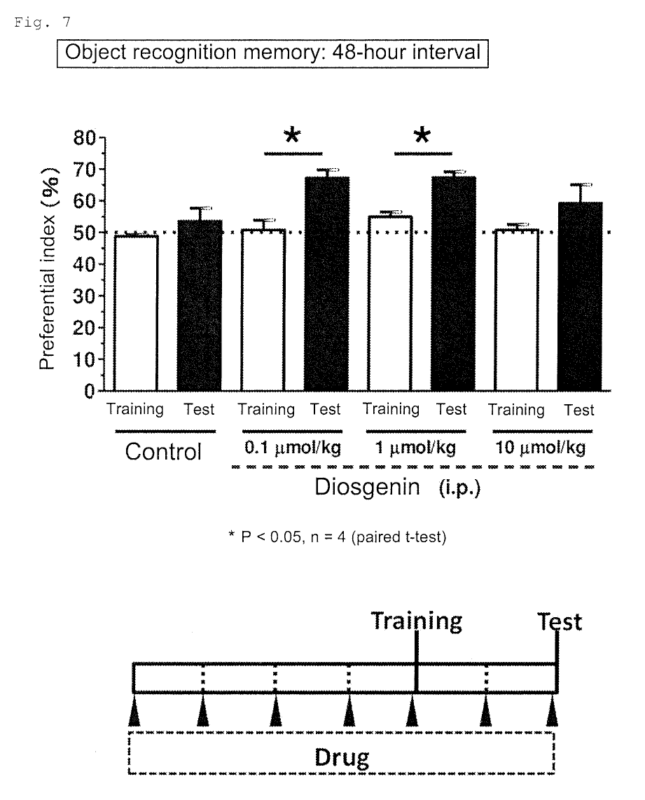

FIG. 7 shows the results. Memory enhancement was observed in the mice of Reference Examples 3 to 5.

The above results were obtained with diosgenin and the particular diosgenin derivatives, but similar results were also found with other diosgenin derivatives. For example, in addition to Dios-F, which is a compound derived from diosgenin by replacement of the hydroxyl group at the 3 position by the fluorine, other diosgenin derivatives were also subjected to docking simulation to 1,25D₃-MARRS. The results revealed that a compound derived from diosgenin by substitution at the 2 (α or β) position with fluorine and a compound derived from diosgenin by substitution at the 4 (α or β) position with fluorine showed a similar binding affinity (kcal/mol) to those of diosgenin and Dios-F. A smaller binding affinity value (kcal/mol) indicates a higher binding activity.

The results of the docking simulation are shown below. In the table below, the term “ds” means “diosgenin”, the term “dsF-30” means a compound derived from diosgenin by replacement of the C3 hydroxyl by fluorine, the term “dsF-2β” means a compound derived from diosgenin by substitution at the 2 (β) position with fluorine, the term “dsF-2α” means a compound derived from diosgenin by substitution at the 2 (α) position with fluorine, the term “dsF-4β” means a compound derived from diosgenin by substitution at the 4 (β) position with fluorine, and the term “dsF-4α” means a compound derived from diosgenin by substitution at the 4 (α) position with fluorine.

TABLE 2

| Compound | Binding affinity (kcal/mol) |
|---|---|
| ds | −9.4 |
| dsF-3β | −9.5 |
| dsF-4β | −9.8 |
| dsF-4α | −9.4 |
| dsF-2β | −9.5 |
| dsF-2α | −9.8 |

### Synthesis Example 1: Synthetic method of Dios-G ((3β,25R)-3-(2-aminoethanoyloxy)-spirost-5-ene hydrochloride)

In CH₂Cl₂ (24.0 mL) were dissolved diosgenin (Wako Pure Chemical Industries, Ltd.) (1.00 g, 2.41 mmol) and Fmoc-Gly-OH (2.15 g, 7.24 mmol). To the solution on ice were successively added EDCI (1-(3-dimethylaminopropyl)-3-ethylcarbodiimide) (1.39 g, 7.24 mmol), DMAP (N,N-dimethyl-4-aminopyridine) (29.4 mg, 0.241 mmol), and i-Pr₂Net (N,N-diisopropylethylamine) (1.12 g, 8.68 mmol). The mixture was stirred at room temperature for 24 hours, and the reaction was stopped by addition of water. Extraction was performed with ethyl acetate (30 mL). The combined organic layer was washed with saturated brine (10 mL), and then dried over magnesium sulfate. The organic solvent was removed by evaporation under reduced pressure. The residue was purified by flash silica gel column chromatography (eluent: hexane/ethyl acetate=70:30) to give diosgenin-Fmoc-glycinate (1.19 g, 71%).

The diosgenin-Fmoc-glycinate (1.19 g, 1.71 mmol) was dissolved in a CH₃CN—CH₂Cl₂ mixed solution (15 mL, 3:2 v/v). To the solution, piperidine (1.46 g, 17.1 mmol) was added at room temperature. The mixture was stirred at room temperature for 1 hour to give a suspension. To the suspension, toluene (10 mL) was added to give a clear solution, and the organic solvent was removed by evaporation under reduced pressure. To the residue, toluene (10 mL) was added to give a solution, and the organic solvent was removed by evaporation under reduced pressure. This operation was repeated once. The residue was purified by flash silica gel column chromatography (eluent: hexane/ethyl acetate=80:20 to 0:100) to give (3β,25R)-3-(2-aminoethanoyloxy)-spirost-5-ene (693 mg, 86%).

The thus obtained (3β,25R)-3-(2-aminoethanoyloxy)-spirost-5-ene (590 mg, 1.25 mmol) was dissolved in Et₂O (50 mL). To the solution on ice, a hydrochloric acid (HCl) solution in diethyl ether (Et₂O) (1.0 M, 3.13 mL, 3.13 mmol) was added dropwise, and the mixture was stirred at room temperature for 30 minutes. The precipitates were collected by filtration to give the title compound (535 mg, 84%) as a white solid. The identification of the compound was carried out by comparing the ¹H NMR data with the reported data (Wang, X., Ye, Z., Wang, L., Faming Zhuanli Shenging Gongkai Shuomingshu (2004), CN 151760 A 20040804).

Mp 194-197° C.; IR (KBr) 3400, 2951, 1746, 1598 cm⁻¹; ¹H NMR (500 MHz, CD₃OD) δ 5.42 (1H, d, J=5.6 Hz), 4.72-4.66 (1H, m), 4.41-4.37 (1H, m), 3.80 (3H, s), 3.46-3.43 (2H, m), 2.43-2.38 (3H, m), 2.06-1.88 (5H, m), 1.79-1.13 (17H, m), 1.08 (3H, s), 0.95 (3H, d, J=7.2 Hz), 0.81 (3H, s), 0.78 (3H, d, J=6.4 Hz); ¹³C NMR (125 MHz, CD₃OD) δ 167.9, 140.6, 123.9, 110.5, 82.1, 77.6, 67.8, 63.7, 57.7, 51.5, 42.9, 41.4, 41.2, 40.8, 38.9, 38.0, 37.9, 33.1, 32.7, 32.4, 31.4, 29.9, 28.6, 21.9, 19.74, 19.70, 17.5, 16.8, 14.9; LRMS (FAB) m/z 508 ([M+H]+); HRMS (FAB) m/z calcd. For C₂₉H₄₇O₄ClN ([M+H]⁺) 508.31936, found 508.31852.

### Synthesis Example 2: Synthetic method of Dios-F ((3β,25R)-3-fluorospirost-5-ene)

In dichloromethane (CH₂Cl₂) (0.63 mL) was suspended XtalFluor-E (registered trademark) (Sigma-Aldrich) (85.9 mg, 0.375 mmol). To the suspension at room temperature, triethylamine tris-hydrofluoride (Et₃N.3HF) (0.16 mL, 1.00 mmol) was added, and then diosgenin (Wako Pure Chemical Industries, Ltd.) (118 mg, 0.25 mmol) was added. The solution was stirred at room temperature for 21 hours. After TLC showed the disappearance of the starting material, the reaction was stopped by addition of a 5% aqueous solution of Na₂CO₃. Extraction with ethyl acetate (1 mL) was performed three times. The combined organic layer was washed with saturated brine (1 mL) and dried over magnesium sulfate. The solid was filtered off, and the organic solvent was removed by evaporation under reduced pressure. The residue was purified by flash silica gel column chromatography (eluent: hexane/ethyl acetate=98:2) to give the title compound (49.3 mg, 47%) as a white solid. The white solid was further recrystallized from ethyl acetate to give 19.1 mg of clear, colorless needle crystals.

Mp 224-226° C.; R_{f}=0.36 (silica gel, hexane/AcOEt, 98:2); ¹H NMR (500 MHz, CDCl₃) δ 5.39 (1H, d, J=4.2 Hz), 4.46-4.39 (1H, m), 4.38 (1H, dm, ²J_{H-F}=50 Hz), 3.43 (1H, dd, J=10.9, 10.9 Hz), 3.38 (1H, dd, J=10.9, 3.1 Hz), 2.46-2.44 (2H, m), 2.03-1.96 (3H, m), 1.90-1.85 (2H, m), 1.79-1.43 (14H, m), 1.36-1.26 (1H, m), 1.21-1.07 (2H, m), 1.04 (3H, s), 0.97 (3H, d, J=6.8 Hz), 0.80-0.78 (6H, m); ¹³C NMR (125 MHz, CDCl₃) δ 139.3 (d, ³J_{C-F}=11.9 Hz), 122.7, 109.3, 92.7 (d, ¹J_{C-F}=173 Hz), 80.8, 66.8, 62.1, 56.4, 49.91, 49.84, 41.6, 40.2, 39.7, 39.3 (d, ²J_{C-F}=19.3 Hz), 36.7, 36.3 (d, ³J_{C-F}=11.0 Hz), 32.0, 31.8, 31.4, 30.3, 28.8, 28.7 (d, ²J_{C-F}=17.3 Hz), 20.9, 19.3, 17.1, 16.3, 14.5; LRMS (EI) m/z 417 [M⁺]; HRMS (EI) m/z calcd. for C₂₇H₄₁FO₂ 416.3091 [M⁺], found 416.3137.

### Example 11

To 12.92 mg of wild yam dried extract (ASK INTERCITY Co., Ltd., containing 16.05% diosgenin) was added 5 mL of soybean oil (KANEDA Co., Ltd.), and the mixture was stirred with a microhomogenizer to give a uniform suspension. A0.5 mL aliquot of the suspension was uniformly mixed with 49.5 mL of soybean oil to give a suspension containing diosgenin at 0.004146 mg/mL in the soybean oil (Example Product 11). Example Product 11 was orally administered to the AD model mice (5XFAD, male and female, 30 to 47 weeks old) once a day at a diosgenin dosage of 0.1 μmol/kg·day per unit weight of the animal. The administration period was 14 days. The mice were then subjected to the object recognition memory test. The training session was performed on the next day of the final administration. The interval between the training session and the test session was 1 hour.

### Comparative Example 6

The administration and the memory test were performed in the same manner as in Example 11 except that soybean oil alone was used instead of Example Product 11.

### Control

The administration and the memory test were performed in the same manner as in Comparative Example 6 except that wild-type mice (34 to 36 weeks old) were used instead of the AD model mice.

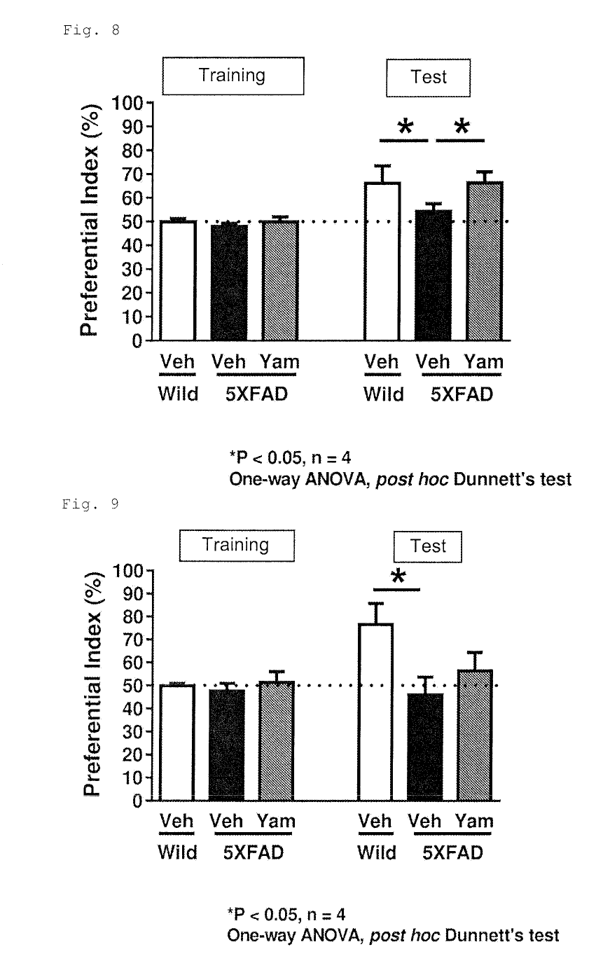

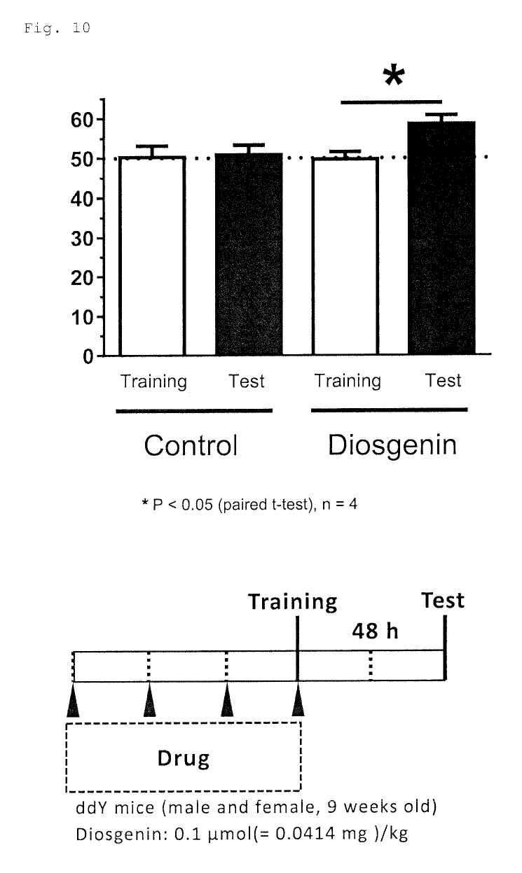

FIG. 8 shows the results. In FIG. 8, the term “Preferential index” indicates the preferential index as described above, the term “Wild” indicates wild-type mice, the term “5XFAD” indicates the AD model mice, the term “Yam” indicates the suspension containing the wild yam dried extract (Example 11), and the term “Veh” indicates the vehicle containing no wild yam dried extract (Comparative Example 6 and the control). The three bars in the left side in the figure show the results in the training session, and the three bars in the right side in the figure show the results in the test session (the same is applied to FIG. 9).

As apparent from the results in FIG. 8, the memory deficits of the mice in Example 11 was improved to be comparable with wild-type mice used as the control.

### Comparative Example 7

To 12.92 mg of wild yam dried extract (ASK INTERCITY Co., Ltd., containing 16.05% diosgenin) was added 5 mL of distilled water, and the mixture was stirred with a microhomogenizer to give a uniform suspension. A 0.5 mL aliquot of the suspension was mixed with 49.5 mL of distilled water to give a suspension containing diosgenin at 0.004146 mg/mL in the distilled water (Example Product 12). Example Product 12 was orally administered to the AD model mice (5XFAD, male, 30 to 47 weeks old) once a day at a diosgenin dosage of 0.1 μmol/kg·day per unit weight of the animal. The administration period was 14 days. The mice were then subjected to the object recognition memory test. The training session was performed on the next day of the final administration. The interval between the training session and the test session was 1 hour.

### Comparative Example 8

The administration and the memory test were performed in the same manner as in Comparative Example 7 except that distilled water was used instead of Example Product 12.

### Control

The administration and the memory test were performed in the same manner as in Comparative Example 8 except that wild-type mice (39 to 43 weeks old) were used instead of the AD model mice.

FIG. 9 shows the results.

As apparent from the results in FIG. 9, the memory deficits was not significantly improved in Comparative Example 7, in which distilled water was used.

### Example 12

To 2.07 mg of diosgenin (Wako Pure Chemical Industries, Ltd.) was added 5 mL of sesame oil (KANEDA Co., Ltd.), and the mixture was stirred with a microhomogenizer to give a uniform suspension. A 0.5 mL aliquot of the suspension was uniformly mixed with 49.5 mL of sesame oil to give a suspension containing diosgenin at 0.004146 mg/mL in the sesame oil (Example Product 13). Example Product 13 was orally administered to ddY mice (male and female, 9 weeks old) once a day at a diosgenin dosage of 0.1 μmol/kg·day per unit weight of the animal. The administration period was 4 days. The mice were then subjected to the object recognition memory test. The training session was performed one hour after the final administration. The interval between the training session and the test session was 48 hours.

### Comparative Example 9

The administration and the memory test were performed in the same manner as in Example 12 except that sesame oil alone was used instead of Example Product 13.

FIG. 10 shows the results. The mice to which the diosgenin suspension in sesame oil was orally administered in Example 12 showed a significant enhancement of the object recognition memory.

### INDUSTRIAL APPLICABILITY

The present invention provides a clinically applicable prophylactic or therapeutic drug effectively used for radical cure of Alzheimer's disease, which has been treated by symptomatic treatment. The present invention also provides a clinically applicable drug for preventing or treating diseases associated with axonal dysfunction other than Alzheimer's disease.

1. An oral drug comprising one or more compounds selected from the group consisting of diosgenin, a diosgenin derivative and a pharmaceutically acceptable salt thereof, wherein the one or more compounds are suspended or dissolved in an oil or fat.

2. The drug according to claim 1, comprising at least diosgenin.

3. The drug according to claim 1, wherein the diosgenin derivative is at least one compound selected from the group consisting of compounds represented by formula (I-1): 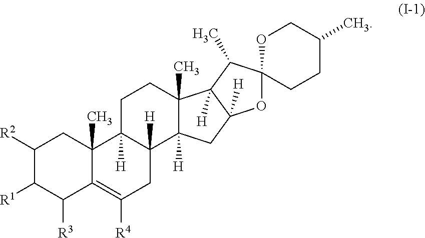
wherein R¹, R², R³, and R⁴ are the same or different and each are a hydrogen atom or a substituent, with the proviso that when R², R³, and R⁴ are a hydrogen atom, R¹ is not a hydroxyl group and
a pharmaceutically acceptable salt thereof.

4. The drug according to claim 3, wherein the substituent in formula (I-1) is a hydrocarbon group, a hydroxyl group, an —O—(CH₂)ₙ—CH₃ group, an —O—(CH₂)ₘ—NH₂ group, an —O—(CH₂)ₘ—COOH group, an —O—(CH₂)ₘ—SO₃H group, an —O—CO—(CH₂)ₙ—CH₃ group, an —O—CO—NH—(CH₂)ₙ—CH₃ group, an —O—CO—NR—(CH₂)ₙ—CH₃ group, an —O—CO—NH—CH(R^{b})—COOH group, an —O—(CH₂)ₙ—CO—NH-AD group wherein AD is an adamantyl group, an —O—CO—NH—(CH₂)ₘ—SO₃H group, an —O—CO—NH—(CH₂)ₘ—COOH group, an —O—CO—O—(CH₂)ₙ—CH₃ group, an —O—CO—S—(CH₂)ₙ—CH₃ group, an —O—SU group wherein SU is a sugar chain, an —O—SO₂—OH group, an —O—PO₂—OH group, an —(OCH₂CH₂)ₘ—CH₃ group, an —(OCH₂CH₂CH₂)ₘ—CH₃ group, a carboxyl group, a —COO(CH₂)ₙCH₃ group, a —CO—NH—(CH₂)ₙ—CH₃ group, an —SO₃H group, an —SO₂—(CH₂)ₙ—CH₃ group, an —SO₂-Ph group wherein Ph is a phenyl group, a —CO—NH—CH(R^{b})—COOH group, a —CO—NH—(CH₂)ₙ—SO₃H group, an amino group, an —NH—(CH₂)ₙ—CH₃ group, an —NH—(CH₂)ₙ—NH₂ group, an —NH—CH(R^{b})—COOH group, an —NH—(CH₂)ₘ—SO₃H group, an —NH—(CH₂)ₘ—SO₂H group, an —NH—CO—O—(CH₂)ₙ—CH₃ group, an —NH—CO—NH₂ group, an —NH—CO—NH-AD group wherein AD is an adamantyl group, an —NH—CO—NH—CH(R^{b})—COOH group, an —NH—CO—NH—(CH₂)ₘ—SO₃H group, an —NH—CO—NH—(CH₂)ₘ—COOH group, a mercapto group, an —S—(CH₂)ₙ—CH₃ group, an —S—(CH₂)ₘ—COOH group, an —S—(CH₂)ₘ—CH(NH₂)—COOH group, an —S—CO—NH-AD group wherein AD is an adamantyl group, an —S—S—(CH₂)ₘ—CH(NH₂)—COOH group, an —SO₃H group, a —PO₃H group, an amino acid group, or a halogen atom,
wherein m is an integer of 1 or more, n is an integer of 0 or more, and R^{b} is a hydrogen atom or a hydrocarbon group.

5. The drug according to claim 1, wherein the diosgenin derivative is one or more compounds selected from the group consisting of (3β,25R)-3-(2-aminoethanoyloxy)-spirost-5-ene, (3β,25R)-3-fluorospirost-5-ene, (3β25R)-3-(2-aminoethylsulfonyloxy)-spirost-5-ene, (3β,25R)-3-(2-aminopropylsulfonyloxy)-spirost-5-ene, (3β,25R)-3-[N-(2,6-dimethyladamantan-1-yl)carbamoyloxy]-spirost-5-ene, (3β,25R)-3-{[N-(2,6-dimethyladamantan-1-yl)carbamoyl]amino}-spirost-5-ene, (3β,25R)-3-[N-(2,6-dimethyladamantan-1-yl)carbamoylthio]-spirost-5-ene, (3β,25R)-3-{[N-(adamantan-1-yl)carbamoyl]amino}-spirost-5-ene, (3β,25R)-3-[N-(adamantan-1-yl)carbamoylthio]-spirost-5-ene, (3β,25R)-3-[N-(adamantan-1-yl)carbamoyloxy]-spirost-5-ene, and pharmaceutically acceptable salts thereof.

6. The drug according to claim 1, which is a prophylactic or therapeutic drug for a disease associated with axonal dysfunction.

7. The drug according to claim 6, wherein the disease associated with axonal dysfunction is Alzheimer's disease.

8. The drug according to claim 6, wherein the disease associated with axonal dysfunction is spinal cord injury.

9. The drug according to claim 1, which is a drug for extending axons.

10. The drug according to claim 1, which is a drug for repairing degenerated axons.

11. The drug according to claim 1, which is a drug for improving memory or a drug for suppressing the deterioration of memory.

12. The drug according to claim 1, which is combined with one or more compounds known to be effective for the treatment or prevention of a disease associated with axonal dysfunction or a pharmaceutically acceptable salt thereof.

13. The drug according to claim 1, which is in one or more dosage forms selected from the group consisting of liquids, suspensions, capsules, soft capsules, tablets, granules, powders, syrups, jellies, orally disintegrating tablets, and chewable tablets.

14. A functional health food comprising the drug according to claim 1.

15. (3β,25R)-3-fluorospirost-ene represented by formula (III):

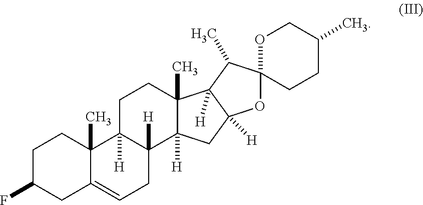

16. A prophylactic or therapeutic drug for a disease associated with axonal dysfunction, a drug for extending axons, a drug for repairing degenerated axons and/or a drug for improving memory or for suppressing the deterioration of memory, the drug comprising at least one compound selected from a diosgenin derivative and a pharmaceutically acceptable salt thereof.

17. The drug according to claim 16, wherein the disease is spinal cord injury.

18-20. (canceled)

21. A functional health food comprising at least one compound selected from a diosgenin derivative and a pharmaceutically acceptable salt thereof or the drug according to claim 16.
